(19) United States
(12) Patent Application Publication
(10) Pub. No.: US 2024/0150816 A1
(43) Pub. Date: May 9, 2024

(54) TARGETED SHRINKING OR EXPANSION OF BIOMOLECULES

(71) Applicant: 10x Genomics, Inc., Pleasanton, CA (US)

(72) Inventors: Hongbo FENG, Dublin, CA (US); Shalini GOHIL, Castro Valley, CA (US); Malte KÜHNEMUND, Stockholm (SE)

(21) Appl. No.: 18/478,248

(22) Filed: Sep. 29, 2023

Related U.S. Application Data
(60) Provisional application No. 63/412,374, filed on Oct. 1, 2022.

(51) Int. Cl. C12Q 1/6816; C12Q 1/6841; C12Q 1/6844

(52) U.S. Cl. CPC C12Q 1/6816; C12Q 1/6841; C12Q 1/6844

(57) ABSTRACT

The present disclosure in some aspects relates to methods and compositions for processing a biological sample, including methods and compositions for targeted shrinking and expansion of biomolecules in a biological sample for in situ analysis. In some aspects, a polymer is conjugated with a probe or product thereof (e.g., rolling circle amplification products) in the biological sample, and the polymer conjugate may be shrunk or expanded. In some aspects, a size-controllable polymer network is used to shrink or expand the biomolecules in the biological sample. Targeted shrinking can create more space for analyzing more biomolecules at the same time inside a confined space within the sample and expand the space between adjacent probes or products thereof at locations in the sample.

### CROSS-REFERENCE TO RELATED APPLICATIONS

This application claims priority to U.S. Provisional Patent Application No. 63/412,374, filed Oct. 1, 2022, entitled “TARGETED SHRINKING OR EXPANSION OF BIOMOLECULES,” which is herein incorporated by reference in its entirety for all purposes.

### FIELD

The present disclosure relates in some aspects to methods and compositions for processing a biological sample, such as methods and compositions for targeted shrinking and expansion of biomolecules in a biological sample for in situ analysis.

### BACKGROUND

Methods are available for processing a biological sample in situ, such as a cell or a tissue. For example, crosslinking may be used to shrink and/or expand a biological sample to increase resolution of individual biomolecules during downstream detection of the biomolecules. However, methods for in situ analysis using crosslinking may result in off-target disruption of molecules and/or structures in the biological sample. Improved methods for in situ analysis are needed. The present disclosure addresses these and other needs.

### BRIEF SUMMARY

There is a need for shrinking or expanding structures comprising target analytes (e.g., biomolecules) in a biological sample, including, for example, shrinking biomolecules (e.g., nucleic acid concatemers such as rolling circle amplification (RCA) products (RCPs) and proteins), expanding biomolecules (e.g., nucleic acids within chromatin regions), and/or stabilizing biomolecules during in situ analysis of a biological sample. One exemplary embodiment of the present disclosure comprises shrinking of nucleic acids (e.g., a nucleic acid probe or product thereof) in the sample (e.g., a cell or tissue sample) which may allow for better resolution of signals and detection of individual nucleic acid molecules. Targeted shrinking of nucleic acids in the sample may compact the structures themselves to occupy less space in a sample, without the need to compact other structures in the sample or compact the entire sample. The shrinking may also be advantageous for non-nucleic acid analytes, including proteins and antibodies, as shrinking expands the space between compacted molecules at adjacent locations in the biological samples. Another exemplary embodiment of the disclosure comprises expansion of densely packed regions that are challenging to analyze, such as chromatin regions in a cell or tissue sample which may allow for better probe binding to these generally inaccessible or less accessible genomic regions.

In some embodiments, provided herein are methods and compositions for shrinking or expanding molecules or structures comprising or associated with target analytes in situ in a biological sample. In particular, the present disclosure provides methods for shrinking or expanding biomolecules prior to and/or during the in situ analysis. In some embodiments, the present disclosure provides methods for decoding identifier sequences (e.g., analyte sequences or barcode sequences) through sequential cycles of detectable probe hybridization (directly or indirectly) to the biomolecules. In some embodiments, the identifier sequences can be analyte sequences or barcode sequences. In some embodiments, the biomolecules can be nucleic acid concatemers such as RCPs, protein, or antibodies. The shrinking or expanding may be achieved through contacting the biological sample with a polymer that is capable of forming a polymer conjugate with the biomolecule but not with other adjacent molecules or structures in the sample. The polymer conjugate can be shrunk or expanded, thereby respectively shrinking or expanding the biomolecule in a targeted manner and without shrinking or expanding the adjacent molecules or structures.

In some aspects, provided herein is a method for processing a biological sample, comprising: (a) contacting the biological sample with a polymer, wherein: the polymer comprises one or more functional groups A; the biological sample comprises a probe or a product thereof that comprises one or more functional groups B; and the probe comprises a nucleic acid and is configured to directly or indirectly bind to a target analyte in the biological sample; (b) reacting the one or more functional groups A with the one or more functional groups B, thereby coupling the polymer to the probe or product thereof to form a polymer conjugate; (c) removing unreacted polymer from the biological sample; and (d) shrinking or expanding the polymer conjugate, thereby respectively shrinking or expanding the probe or product thereof.

In some embodiments, removing unreacted polymer from the biological sample may be performed by washing the sample. In any of the preceding embodiments, the method may further comprise initiating polymer-polymer cross-linking to form a polymer network between the polymers of the polymer conjugate.

In any of the preceding embodiments, the reaction between functional group A and functional group B may form a covalent bond. In any of the preceding embodiments, the reaction between functional group A and functional group B may be a click reaction. In any of the preceding embodiments, the click reaction between functional group A and functional group B may be biorthogonal. In some embodiments, the click reactions between functional groups A and functional groups B may be orthogonal to one or more enzymatic reactions in the sample. In any of the preceding embodiments, the functional group A and functional group B pair may be selected from the group consisting of: (i) 3′-azido/5′-alkynyl; (ii) 3′-alkynyl/5′-azido; (iii) 3′-azido/5′-cyclooctynyl; (iv) 3′-cyclooctynyl/5′-azido; (v) 3′-tetrazine/5′-dienophile; (vi) 3′-dienophile/5′-tetrazine; (vii) 3′-thiol/5′-alkynyl; (viii) 3′-alkynyl/5′-thiol; (ix) 3′-cyano/5′-1,2-amino thiol; (x) 3′-1,2-amino thiol/5′-cyano; (xi) 3′-nitrone/5′-cyclooctynyl; and (xii) 3′-cyclooctynyl/5′-nitrone.

In any of the preceding embodiments, the target analyte may be a non-nucleic acid target analyte and the probe comprises a target-binding moiety and a reporter oligonucleotide, wherein the target-binding moiety may directly or indirectly bind to the non-nucleic acid target. In some embodiments, the target analyte may be a target protein and the target-binding moiety may be an antibody or epitope binding fragment thereof. In any of the preceding embodiments, the one or more functional groups B may be in the target-binding moiety and/or the reporter oligonucleotide. In any of the preceding embodiments, shrinking the probe or product thereof may shrink the non-nucleic acid target analyte.

In any of the preceding embodiments, the target analyte may be a nucleic acid target analyte. In any of the preceding embodiments, the biological sample may comprise a plurality of primary probes each hybridized to a target sequence in the target nucleic acid. In any of the preceding embodiments, each of the plurality of primary probes or a product or complex thereof may independently comprise one or more functional groups B. In any of the preceding embodiments, the product or complex of the primary probe may be selected from the group consisting of: a rolling circle amplification (RCA) product (RCP), a complex comprising an initiator and an amplifier for hybridization chain reaction (HCR), a complex comprising an initiator and an amplifier for linear oligonucleotide hybridization chain reaction (LO-HCR), a primer exchange reaction (PER) product, and a complex comprising a pre-amplifier and an amplifier for branched DNA (bDNA). In any of the preceding embodiments, the product of the primary probe may be an RCP and nucleotides comprising one or more functional groups B may be incorporated into the RCP during RCA, and wherein the RCP may be shrunk. In any of the preceding embodiments, the primary probe may be selected from the group consisting of: a primary probe comprising a 3′ or 5′ overhang upon hybridization to the target sequence. In some embodiments, the 3′ or 5′ overhang may comprise one or more barcode sequences; a primary probe comprising a 3′ overhang and a 5′ overhang upon hybridization to the target sequence. In some embodiments, the 3′ overhang and the 5′ overhang each may independently comprise one or more barcode sequences; a circular primary probe; a circularizable primary probe or probe set; a primary probe or probe set comprising a split hybridization region configured to hybridize to a splint. In some embodiments, the split hybridization region may comprise one or more barcode sequences; and a combination thereof.

In any of the preceding embodiments, the biological sample may comprise a plurality of intermediate probes that directly or indirectly bind to a primary probe or a product or complex thereof, wherein the primary probe may hybridize to a target sequence in the target nucleic acid. In any of the preceding embodiments, each of the plurality of intermediate probes or a product or complex thereof may independently comprise one or more functional groups B. In any of the preceding embodiments, the product or complex of the intermediate probe may be selected from the group consisting of: a rolling circle amplification (RCA) product (RCP), a complex comprising an initiator and an amplifier for hybridization chain reaction (HCR), a complex comprising an initiator and an amplifier for linear oligonucleotide hybridization chain reaction (LO-HCR), a primer exchange reaction (PER) product, and a complex comprising a pre-amplifier and an amplifier for branched DNA (bDNA). In any of the preceding embodiments, the product of the intermediate probe may be an RCP and nucleotides comprising one or more functional groups B may be incorporated into the RCP during RCA, and wherein the RCP may be shrunk. In any of the preceding embodiments, the intermediate probe may be selected from the group consisting of: an intermediate probe comprising a 3′ or 5′ overhang upon binding to the primary probe or product or complex thereof. In some embodiments, the 3′ or 5′ overhang may comprise one or more barcode sequences; an intermediate probe comprising a 3′ overhang and a 5′ overhang upon binding to the primary probe or product or complex thereof. In some embodiments, the 3′ overhang and the 5′ overhang each may independently comprise one or more barcode sequences; a circular intermediate probe; a circularizable intermediate probe or probe set; an intermediate probe or probe set comprising a split hybridization region configured to hybridize to a splint. In some embodiments, the split hybridization region may comprise one or more barcode sequences; and a combination thereof.

In any of the preceding embodiments, the probe or product thereof may comprise a detectable label.

In any of the preceding embodiments, the probe or product thereof may comprise a region that directly or indirectly binds to a detectably labeled probe. In any of the preceding embodiments, the probe or product thereof may comprise a region that directly or indirectly binds to a compaction oligonucleotide. In any of the preceding embodiments, the compaction oligonucleotide may comprise two or more hybridization regions that hybridize to compaction regions in the probe or product thereof. In some embodiments, the compaction oligonucleotide may comprise a spacer region between the two or more hybridization regions.

In any of the preceding embodiments, a sequence of the probe or product thereof may be analyzed in situ in the biological sample. In any of the preceding embodiments, the sequence of the probe or product thereof may be analyzed by sequential hybridization, sequencing by hybridization, sequencing by ligation, sequencing by synthesis, sequencing by binding, or a combination thereof. In any of the preceding embodiments, the sequence that is analyzed may comprise a barcode sequence. In some embodiments, the barcode sequence may correspond to an analyte or a portion thereof or a labelling agent for an analyte or a portion thereof in the biological sample.

In any of the preceding embodiments, the method may further comprise detecting a signal associated with the probe or product thereof or the target analyte. In any of the preceding embodiments, the signal may be detected by imaging the biological sample using fluorescent microscopy. In any of the preceding embodiments, the signal may be detected after the shrinking or expansion of the probe or product thereof.

In any of the preceding embodiments, the signal can be detected after the shrinking of the probe or product thereof and the polymer conjugate can be expanded after the signal is detected, thereby expanding the probe or product thereof.

In any of the preceding embodiments, the polymer conjugate can be expanded in a buffer composition comprising at least or about 50% DMSO, a detergent or surfactant, and a salt.

In any of the preceding embodiments, the polymer may be a synthetic polymer, a natural polymer, or combination thereof. In any of the preceding embodiments, the polymer may be selected from the group consisting of poly(acrylate), poly(alkyl-acrylate), poly(hydroxyalkyl-acrylate), poly(acrylamide), poly(alkyl-acrylamide), poly(ethylene glycol), poly(vinyl alcohol), poly(vinyl pyrrolidone), poly(lactic acid), poly(lactic-co-glycolic acid), hyaluronic acid, chitosan, heparin, alginate, fibrin, collagen, gelatin, and copolymers of any two or more of the foregoing.

In any of the preceding embodiments, the polymer-polymer cross-linking may create a physically cross-linked polymer network. In any of the preceding embodiments, the physical cross-linking may occur through ion-ion interactions or hydrogen bonding.

In any of the preceding embodiments, the polymer-polymer cross-linking may create a chemically cross-linked polymer network. In any of the preceding embodiments, the chemically cross-linked polymer network may be created through photo-cross-linking of the polymers. In any of the preceding embodiments, the method may further comprise adding a photoinitiator. In any of the preceding embodiments, the photoinitatior may be selected from the group consisting of: benzoyl peroxide, 2,2-dimethoxy-2-phenylacetophenone, 2-hydroxy-2-methyl-1-phenylpropanone, 1-hydroxy-cyclohexylphenylketone, benzophenone, isopropyl thioxanthone, and an organic peroxide. In any of the preceding embodiments, the chemically cross-linked polymer network may be created by reacting the polymers of the polymer conjugate with a cross-linking reagent. In any of the preceding embodiments, the cross-linking reagent may be selected from the group consisting of a bivalent or trivalent: thiol, epoxide, amine, alcohol, hydrazide, hydroxylamine, and isocyanate. In any of the preceding embodiments, the chemically cross-linked polymer network may be created by a click reaction between the polymers of the polymer conjugate.

In any of the preceding embodiments, the click reaction between the polymers of the polymer conjugate may be biorthogonal. In some embodiments, the click reaction between the polymers of the polymer conjugate may be orthogonal to one or more enzymatic reactions in the sample. In any of the preceding embodiments, the functional groups that participate in the click reaction between the polymers of the polymer conjugate may be selected from the group consisting of: (i) 3′-azido/5′-alkynyl; (ii) 3′-alkynyl/5′-azido; (iii) 3′-azido/5′-cyclooctynyl; (iv) 3′-cyclooctynyl/5′-azido; (v) 3′-tetrazine/5′-dienophile; (vi) 3′-dienophile/5′-tetrazine; (vii) 3′-thiol/5′-alkynyl; (viii) 3′-alkynyl/5′-thiol; (ix) 3′-cyano/5′-1,2-amino thiol; (x) 3′-1,2-amino thiol/5′-cyano; (xi) 3′-nitrone/5′-cyclooctynyl; and (xii) 3′-cyclooctynyl/5′-nitrone. In any of the preceding embodiments, the click reaction between the polymers of the polymer conjugate may be orthogonal to the click reaction between functional group A and functional group B.

In any of the preceding embodiments, shrinking or expanding the polymer conjugate may be initiated by contacting the biological sample with a solution. In any of the preceding embodiments, the solution may be selected from the group consisting of: water, aqueous inorganic acid, aqueous organic acid, aqueous inorganic base, aqueous organic base, aqueous organic salt solution, and aqueous inorganic salt solution.

In any of the preceding embodiments, shrinking or expanding the polymer conjugate may be initiated by changing the temperature of the biological sample.

In any of the preceding embodiments, the polymer may be photosensitive or may comprise a photosensitive functional group. In any of the preceding embodiments, shrinking or expanding the polymer conjugate may be initiated by exposing the biological sample to light. In any of the preceding embodiments, exposing the biological sample to light may be performed prior to and/or during imaging. In any of the preceding embodiments, the light may be used to image the biological sample and/or detect a signal associated with the probe or product thereof or the target analyte.

In any of the preceding embodiments, the biological sample may be a non-homogenized tissue sample or a tissue section. In any of the preceding embodiments, the biological sample may be a processed or cleared biological sample. In any of the preceding embodiments, the method may further comprise processing or clearing the biological sample. In any of the preceding embodiments, contacting the biological sample with the polymer may occur after processing or clearing the biological sample. In any of the preceding embodiments, contacting the biological sample with the polymer may occur before processing or clearing the biological sample. In any of the preceding embodiments, contacting the biological sample with the polymer may occur during processing or clearing the biological sample.

In any of the preceding embodiments, the biological sample may be embedded in a matrix. In some embodiments, the matrix may be a hydrogel. In any of the preceding embodiments, the target analyte may comprise one or more functional groups C for attachment to the matrix. In any of the preceding embodiments, the target analyte may attached to the matrix. In any of the preceding embodiments, the polymer may comprise one or more functional groups D for attachment to the matrix. In any of the preceding embodiments, the polymer may be attached to the matrix.

In any of the preceding embodiments, the probe or product thereof may be conjugated to multiple polymer molecules.

In any of the preceding embodiments, the target analyte may be detected in situ in the biological sample. In any of the preceding embodiments, the method may comprise imaging the biological sample to detect the target analyte. In some embodiments, the imaging may comprise fluorescent microscopy. In any of the preceding embodiments, shrinking or expanding the polymer conjugate may occur during the imaging.

In any of the preceding embodiments, the polymer conjugate can be expanded before or during contacting the biological sample with nucleic acid probes, thereby facilitating direct or indirect binding of the nucleic acid probes to the probe or product thereof.

In any of the preceding embodiments, the polymer conjugate can be shrunk before or during imaging the biological sample to detect signals of nucleic acid probes that directly or indirectly bind to the probe or product thereof.

In any of the preceding embodiments, the polymer conjugate can be expanded after the imaging, thereby facilitating removal of the nucleic acid probes from the probe or product thereof and/or direct or indirect binding of additional nucleic acid probes to the probe or product thereof.

In any of the preceding embodiments, the polymer conjugate can be shrunk before or during imaging the biological sample to detect signals of the additional nucleic acid probes that directly or indirectly bind to the probe or product thereof.

In any of the preceding embodiments, the product can be a rolling circle amplification product.

In any of the preceding embodiments, the biological sample may be a tissue slice between about 1 μm and about 50 μm in thickness. In some embodiments, the tissue slice may be between about 5 μm and about 35 μm in thickness. In any of the preceding embodiments, the biological sample may be a formalin-fixed, paraffin-embedded (FFPE) sample, a frozen tissue sample, or a fresh tissue sample.

In any of the preceding embodiments, the method may comprise shrinking the target analyte in the biological sample. In any of the preceding embodiments, the target analyte may be in a nucleic acid product. In any of the preceding embodiments, the product may be a nucleic acid concatemer. In any of the preceding embodiments, contacting the biological sample with the polymer may occur before generation of the nucleic acid product in the biological sample. In any of the preceding embodiments, contacting the biological sample with the polymer may occur during the generation of the nucleic acid product in the biological sample. In any of the preceding embodiments, contacting the biological sample with the polymer may occur after generation of the nucleic acid product in the biological sample. In any of the preceding embodiments, the nucleic acid product may be generated in situ in the biological sample. In any of the preceding embodiments, the nucleic acid product may be immobilized in the biological sample. In any of the preceding embodiments, the nucleic acid product may be a product of an endogenous nucleic acid molecule in the biological sample. In some embodiments, the product may be an extension product, a replication product, a reverse transcription product, and/or a rolling circle amplification (RCA) product (RCP). In some embodiments, the endogenous nucleic acid molecule may be a viral or cellular DNA or RNA. In some embodiments, the endogenous nucleic acid molecule may be genomic DNA/RNA, mRNA, or cDNA. In any of the preceding embodiments, the nucleic acid product may be a product of a labelling agent that directly or indirectly binds to the target analyte in the biological sample. In some embodiments, the product may be an extension product, a replication product, a reverse transcription product, and/or a rolling circle amplification (RCA) product (RCP). In any of the preceding embodiments, the labelling agent may comprise a reporter oligonucleotide. In some embodiments, the reporter oligonucleotide may comprise one or more barcode sequences and the nucleic acid product may comprise one or a plurality of copies of the one or more barcode sequences. In any of the preceding embodiments, the nucleic acid product may be a rolling circle amplification (RCA) product (RCP) of a circular or circularizable probe or probe set that hybridizes to a DNA or RNA molecule in the biological sample. In any of the preceding embodiments, the nucleic acid product generated from a plurality of different mRNA and/or cDNA molecules may be analyzed, a barcode sequence in a particular circular or circularizable probe or probe set may uniquely correspond to a particular mRNA or cDNA molecule, and the particular circular or circularizable probe or probe set may further comprise an anchor sequence that is common among circular or circularizable probes or probe sets for a subset of the plurality of different mRNA and/or cDNA molecules. In any of the preceding embodiments, the nucleic acid product may be in the form of a nanoball having a diameter of between about 0.1 μm and about 3 μm prior to shrinking the polymer conjugate. In some embodiments, the diameter may be between about 0.1 μm and about 0.5 μm, between about 0.5 μm and about 1 μm, between about 0.8 μm and about 1.3 μm, or between about 1 μm and about 1.5 μm, prior to shrinking the polymer conjugate. In any of the preceding embodiments, the nucleic acid product may be between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length, prior to shrinking the polymer conjugate. In any of the preceding embodiments, the nucleic acid product may be in the form of a nanoball having a diameter between about 0.1-fold and about 20-fold smaller after shrinking the polymer conjugate compared to the diameter prior to shrinking the polymer conjugate.

In any of the preceding embodiments, the method may comprise expanding the target analyte in the biological sample. In any of the preceding embodiments, the target nucleic analyte may be a target nucleic acid in a chromatin region. In any of the preceding embodiments, the target analyte may be a target nucleic acid in an open chromatin region, and expansion of the probe or product thereof may open the open chromatin region. In any of the preceding embodiments, the target analyte may be in a target nucleic acid in a closed chromatin region, and expansion of the probe or product thereof may open the closed chromatin region.

In any of the preceding embodiments, the method may further comprise degrading the polymer conjugate. In any of the preceding embodiments, the degrading may comprise contacting the biological sample with a stripping buffer. In some embodiments, the stripping buffer may comprise a reducing agent. In some embodiments, the reducing agent may be dithiothreitol (DTT). In any of the preceding embodiments, the degrading may comprise changing the temperature of the biological sample. In any of the preceding embodiments, the degrading may comprise exposing the biological sample to light. In any of the preceding embodiments, the degrading may be performed before or after detecting a signal associated with the probe or product thereof or the target analyte in the biological sample. In any of the preceding embodiments, the degrading may be performed simultaneously with removing a detectable probe from the probe or product thereof or the target analyte. In any of the preceding embodiments, the method may further comprise repeating steps (a)-(d) after the degrading and removing steps. In any of the preceding embodiments, the method may further comprise contacting the biological sample with one or more detectable probes after repeating steps (a)-(d), wherein the one or more detectable probes may directly or indirectly bind to the probe or product thereof or the target analyte. In any of the preceding embodiments, the method may further comprise detecting signal(s) associated with the one or more detectable probes in the biological sample.

In some aspects, provided herein is a method for processing a biological sample, comprising: (a) contacting the biological sample with a polymer, wherein: the polymer comprises one or more functional groups A; the biological sample comprises a probe or a product thereof that comprises one or more functional groups B; and the probe comprises a nucleic acid and is configured to directly or indirectly bind to a nucleic acid target analyte in the biological sample; (b) reacting the one or more functional groups A with the one or more functional groups B, thereby coupling the polymer to the probe or product thereof to form a polymer conjugate; (c) removing unreacted polymer from the biological sample; and, (d) shrinking the polymer conjugate, thereby shrinking the probe or product thereof.

In some aspects, provided herein is a method for processing a biological sample, comprising: (a) contacting the biological sample with a polymer, wherein: the polymer comprises one or more functional groups A; the biological sample comprises a probe or a product thereof that comprises one or more functional groups B; and the probe comprises a nucleic acid and is configured to directly or indirectly bind to a nucleic acid target analyte in the biological sample; (b) reacting the one or more functional groups A with the one or more functional groups B, thereby coupling the polymer to the probe or product thereof to form a polymer conjugate; (c) removing unreacted polymer from the biological sample; and, (d) expanding the polymer conjugate, thereby expanding the probe or product thereof. In some embodiments, the nucleic acid target analyte may be in a chromatin region.

In some aspects, provided herein is a method for processing a biological sample, comprising: (a) contacting the biological sample with a polymer, wherein: the polymer comprises one or more functional groups A; the biological sample comprises a probe or a product thereof that comprises one or more functional groups B; and the probe comprises a nucleic acid and is configured to directly or indirectly bind to a protein target analyte in the biological sample; (b) reacting the one or more functional groups A with the one or more functional groups B, thereby coupling the polymer to the probe or product thereof to form a polymer conjugate; (c) removing unreacted polymer from the biological sample; and, (d) shrinking the polymer conjugate, thereby shrinking the probe or product thereof.

In some aspects, provided herein is a method for processing a biological sample, comprising: (a) contacting the biological sample with a polymer, wherein: the polymer comprises one or more functional groups A; the biological sample comprises a product of a probe that comprises one or more functional groups B; the probe comprises a nucleic acid and is configured to directly or indirectly bind to a nucleic acid target analyte in the biological sample; and the product is a nucleic acid concatemer; (b) reacting the one or more functional groups A with the one or more functional groups B, thereby coupling the polymer to the probe or product thereof to form a polymer conjugate; (c) removing unreacted polymer from the biological sample; and, (d) shrinking the polymer conjugate, thereby shrinking the nucleic acid concatemer. In some embodiments, the nucleic acid concatemer may be an RCP.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows an exemplary method for shrinking a biomolecule 102 (e.g., a nucleic acid concatemer) comprising functional group B in a biological sample, by applying a polymer 104 (or a cross-linked polymer network 106) comprising a plurality of functional group A that reacts with functional group B to form a polymer conjugate 108. The polymer conjugate is shrunk to form a shrunken conjugate 110, thereby shrinking the biomolecule therein. The polymer can be a linear molecule or a branched molecule.

FIG. 2 shows an exemplary method for shrinking a biomolecule 202 (e.g., a nucleic acid concatemer) comprising functional group B in a biological sample, by applying a polymer 204 comprising functional group A that reacts with functional group B to form a polymer conjugate 206. Polymers of the polymer conjugate are cross-linked to form a polymer network conjugated biomolecule 208, which is shrunk to form a shrunken conjugate 210.

FIG. 3 shows an exemplary method for expanding a biomolecule 302 (e.g., a nucleic acid at or near a chromatin region) comprising functional group B in a biological sample, by applying a polymer 304 (or a cross-linked polymer network 306) comprising a plurality of functional group A that reacts with functional group B to form a polymer conjugate 308. The polymer conjugate is expanded to form an expanded conjugate 310, thereby expanding the biomolecule therein. The polymer can be a linear molecule or a branched molecule.

FIG. 4 shows an exemplary method for expanding a biomolecule 402 (e.g., a nucleic acid at or near a chromatin region) comprising functional group B in a biological sample, by applying a polymer 404 comprising functional group A that reacts with functional group B to form a polymer conjugate 406. Polymers of the polymer conjugate are cross-linked to form a polymer network conjugated biomolecule 408, which is expanded to form an expanded conjugate 410.

FIG. 5 depicts an exemplary method for shrinking a biomolecule in a tissue sample.

FIG. 6 shows schematics showing the signal intensity (I) as a function of RCP size (R). The illustrated RCP coil and detectably labeled probes on the left depict that probes have full access to bind the RCP, where I˜R\hat{ }2. The illustrated RCP coil and detectably labeled probes on the right depict that probes only have access to bind the surface of the RCP, where I˜R.

FIGS. 7A-7B illustrate the relationship between RCP brightness (e.g., intensity I) and RCP size in different sample embedding conditions (FIG. 7A), and the RCP accessibility parameters in different sample embedding conditions (FIG. 7B).

### DETAILED DESCRIPTION

All publications, comprising patent documents, scientific articles and databases, referred to in this application are incorporated by reference in their entireties for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

When utilizing in situ analysis, proper analysis of samples densely packed with biomolecules (such as nucleic acid concatemers and/or proteins) may depend upon resolution of the individual biomolecules, e.g., in a cell or tissue sample. Detection of biomolecules may be enhanced by compaction of the biomolecules without compacting adjacent structures or compacting the entire sample, as reduction in size of a biomolecule can result in increased local concentration of detectable probes targeting the biomolecule, thereby increasing signal intensity. Target compaction can also allow for increased spatial resolution of individual biomolecules from adjacent biomolecules in the sample. Alternatively, detection of biomolecules in a biological sample may be enhanced by targeted expansion, e.g., by expanding structures comprising biomolecules therein, thereby rendering the biomolecules more accessible to detectable probes.

Existing strategies for shrinking and/or expansion of biomolecules using crosslinking may result in shrinking or expansion of the entire biological sample, resulting in problems for downstream analyses (e.g., detection of target analytes). For example, strategies that draw biomolecules in close proximity by shrinking adjacent molecules or structures or the entire sample may decrease the ability to resolve signals associated with the biomolecules. Thus, there is a need for methods and compositions for targeted shrinking of biomolecules such that individual biomolecules occupy less space in the biological sample and the spacing between adjacent biomolecules increases. Similarly, targeted expansion can open a region of the biological sample to allow better access and detection of biomolecules in the region, without the need to cause global disruption of the biological sample.

Provided herein in some aspects are in situ methods of targeted shrinking or expanding biomolecules using a polymer, allowing better resolution of detectable signals and resolution of individual biomolecules at multiple locations (e.g., adjacent locations) in the biological sample. In some embodiments, nucleic acid probes targeting analytes of interest are utilized, and the nucleic acid probes or products thereof (e.g., RCPs) may comprise a functional group that specifically reacts with a corresponding functional group on exogenously added polymers, resulting in a polymer conjugate. The polymer conjugate may be subsequently shrunk or expanded.

Targeted shrinking of biomolecules (e.g., nucleic acid concatemers such as RCPs, proteins, etc.) in a biological sample via crosslinking without off-target effects (e.g., global shrinking or expansion of the biological sample) in situ may provide a number of advantages. In some aspects, shrinking of biomolecules, such as nucleic acid concatemers, by polymer-polymer crosslinking (e.g., shrinking of polymer conjugates) results in compacted biomolecules. For example, shrunken nucleic acid concatemers may enhance concatemer detection, as reduction in size may increase local concentration of the detectable probes to increase signal intensity. In some instances, the shrinking occurs within each of the biomolecules (e.g., nucleic acid concatemers such as RCPs). Targeted shrinking may also increase positional stability of nucleic acid concatemers in a biological sample in situ, and increase signal resolution during detection by forming discrete puncta spaced from adjacent puncta. Targeted shrinking of non-nucleic acid target analytes (e.g., proteins) can likewise provide increased spatial separation of analytes and improved signal resolution during analysis.

In some embodiments, targeted shrinking and expansion or loosening of biomolecules (e.g., nucleic acid concatemers such as RCPs) with a polymer conjugate or network disclosed herein can be used to control the timing of when biomolecules in a biological sample shrink and expand or loosen up. In some embodiments, nucleic acid probes hybridizing to an RCP can be used to detect the RCP at its location in the biological sample, and prior to and/or during nucleic acid probe hybridization, a buffer that promotes polymer conjugate or network expanding or loosening can be used. For instance, the buffer can comprise a higher DMSO concentration to promote polymer conjugate or network expansion or loosening, and to facilitate nucleic acid probe hybridization. In some embodiments, prior to and/or during the detection (e.g., by imaging the sample using fluorescent microscopy) of signals associated with the nucleic acid probe hybridized to the RCP, a buffer that promotes polymer conjugate or network shrinking can be used. For instance, the buffer can comprise a higher water content to promote polymer conjugate or network shrinking, thereby reducing the size of the RCP, such that the intensity of signals associated with nucleic acid probe molecules hybridized to the RCP can be increased. In some embodiments, after signal detection, a buffer that promotes polymer conjugate or network expanding or loosening can be used to facilitate stripping of nucleic acid probe molecules hybridized to the RCP and hybridization of additional nucleic acid probes for subsequent cycles of probe hybridization to the RCP and signal detection. In some embodiments, a method disclosed herein can be used to reduce RCP size while maintaining signal brightness (e.g., intensity of signals associated with detectably labelled probes that directly or indirectly bind to the RCP) through enhanced probe accessibility to the nucleic acid sequences in the RCP, e.g., by targeted expansion or loosening and shrinking of the RCP in each of one or more cycles of probe hybridization, signal detection (e.g., by imaging the sample), stripping of probes, and rehybridization of probes for the next cycle.

In some embodiments, the shrinking and expansion or loosening capability of the polymer conjugate or network under different stimuli such as buffer conditions, temperature, and light can be controlled through the use of monomer selections, the ratio of two or more monomer species, and/or the mixing ratio of the crosslinkers.

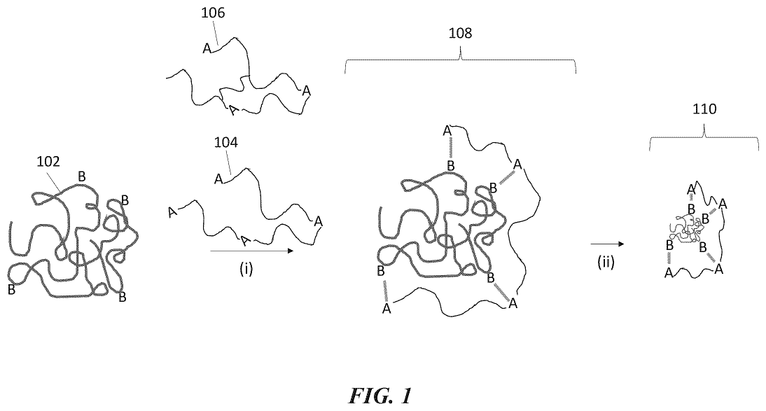

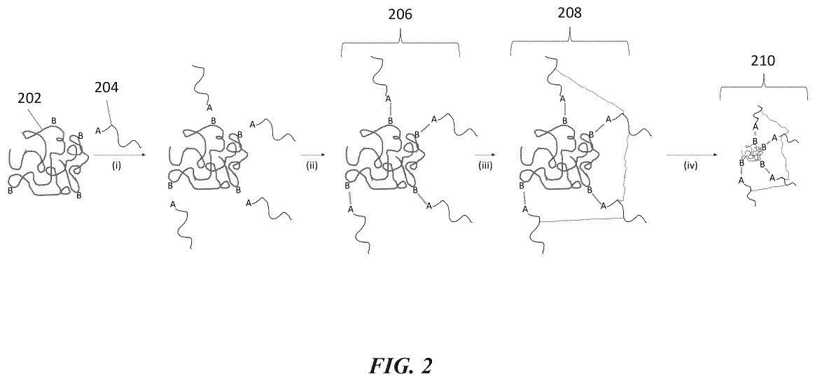

FIGS. 1 and 2 show exemplary workflows for in situ targeted shrinking of a biomolecule in a biological sample. Briefly, a biological sample is contacted with a polymer comprising one or more functional groups A. The polymer may be in the form of a single polymer (e.g., polymer 204 in FIG. 2) or the polymer may be in the form of a pre-crosslinked polymer network (e.g., polymer 104 in a polymer network 106 in FIG. 1). The cross-linked polymer network may be formed by covalent interactions and/or non-covalent interactions, such as ion-ion interactions, hydrogen bonding, Van der Waals interactions, photo-crosslinking of the polymers, chemically crosslinking the polymers using a crosslinking reagent, and/or by a click reaction between the polymers.

In some embodiments, the biological sample comprises a biomolecule (e.g., a nucleic acid concatemer) that comprises one or more functional groups B. The biomolecule can comprise a nucleic acid, which may be a probe that directly or indirectly binds to a target analyte in the biological sample. In some embodiments, the target analyte is a nucleic acid target analyte. In some embodiments, the nucleic acid can be a product of a target nucleic acid or a product of a probe that targets the target nucleic acid. In some embodiments, the nucleic acid can be a nucleic acid probe that hybridizes to a cellular DNA or RNA. In some embodiments, the nucleic acid can be a cDNA of a cellular RNA. In some embodiments, the product of the probe is a nucleic acid product, e.g., a nucleic acid concatemer, such as an RCP. In some embodiments, the nucleic acid can be an RCP of a nucleic acid probe targeting a cellular DNA or RNA or an RCP of a cDNA. In some embodiments, the target analyte is a non-nucleic acid target analyte, such as a protein. In some embodiments, the one or more functional groups A of the polymer are reacted with the one or more functional groups B of the biomolecule to form a polymer conjugate. The reaction between the one or more functional groups A and the one or more functional groups B may form a covalent bond such as one formed via a click reaction. Unreacted polymer can be subsequently removed from the biological sample (e.g., by washing the biological sample), and the polymer conjugate is shrunk, thereby shrinking the biomolecule. Shrinking of the polymer conjugate may be initiated by contacting the biological sample with a solution, changing the temperature of the biological sample, and/or exposing the biological sample to light. Detection and downstream analysis (e.g., decoding) can then be carried out on the smaller biomolecule. Targeted shrinking of the biomolecules allows them to separate into discrete puncta to improve resolution and increase signal intensity, e.g., the fluorophores on the detectable probes have increased local concentration on a smaller RCP.

Expansion of biomolecules in a biological sample via crosslinking without off-target effects in situ may also provide advantages in certain applications. In some aspects, expansion may reduce the density of a region, or increase the physical separation between two or more regions in a biological sample, thereby allowing increase access to analytes in or between the regions. Expansion of a region by polymer-polymer crosslinking may result in an expanded region that enhances analyte access and detection without having to expand the entire sample. For example, a region of DNA that is otherwise inaccessible for analysis, such as a chromatin region, may be expanded according to the methods provided herein to permit access to a target analyte (e.g., nucleic acid sequence) within the chromatin region. In some cases, selective expansion may aid in the detection of molecular interactions (e.g., protein-protein interactions) that may be too close in proximity to detect the analyte(s) and interactions thereof.

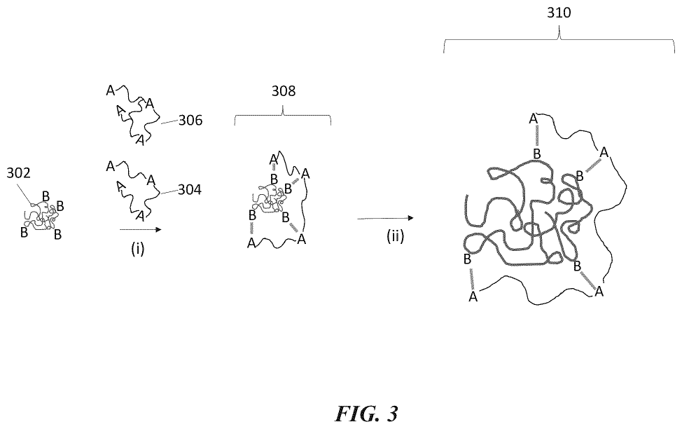

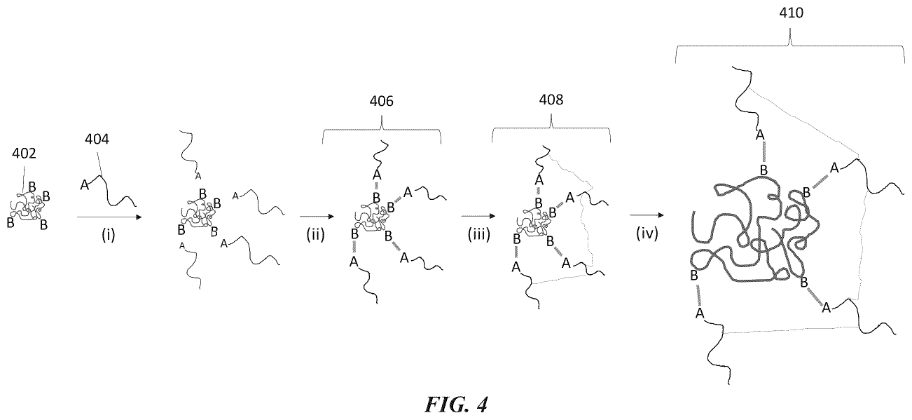

FIGS. 3 and 4 show exemplary workflows for in situ targeted expansion of a biomolecule in a biological sample. Briefly, a biological sample is contacted with a polymer comprising one or more functional groups A. The polymer may be in the form of a single polymer (e.g., polymer 404 in FIG. 4) or the polymer may be in the form of a pre-crosslinked polymer network (e.g., polymer 304 in a polymer network 206 in FIG. 3). The cross-linked polymer network may be formed by covalent interactions and/or non-covalent interactions, such as ion-ion interactions, hydrogen bonding, Van der Waals interactions, photo-crosslinking of the polymers, chemically crosslinking the polymers using a crosslinking reagent, and/or by a click reaction between the polymers. In some embodiments, the target analyte is a nucleic acid sequence that is in a relatively inaccessible region, such as a chromatin region. One or more probes (e.g., nucleic acid probes or nucleic acid analog probes such as PNA or LNA) each comprising one or more functional groups B can be utilized to target the relatively inaccessible region, and the functional groups B can be reacted with the one or more functional groups A of a polymer or polymer network. Unreacted polymer can be subsequently removed from the biological sample (e.g., by washing the biological sample), and the polymer conjugate is expanded, thereby expanding the region. Expansion of the polymer conjugate may be initiated by contacting the biological sample with a solution, changing the temperature of the biological sample, and/or exposing the biological sample to light. Expansion of the polymer conjugate and the one or more probes therein can expose the target nucleic acid sequence, which may be detected via hybridization of detectable probes which would not have access to the target nucleic acid sequence prior to expansion of the relatively inaccessible region.

Overall, the described herein are improved methods for processing a biological sample comprising a biomolecule, which may have applications for in situ analysis of biomolecules. In the following sections, additional description of various aspects of the methods, compositions, and kits disclosed herein is provided. Section II describes methods for in situ targeted shrinking and expansion of biomolecules using a polymer; Section III describes exemplary methods for detection and analysis of the biomolecules; and Section IV describes exemplary biological samples as well as analytes (e.g., endogenous analytes, labelling agents, or products of endogenous nucleic analytes) that can be detected using a method of analyzing a biological sample described herein (e.g., by detecting a target nucleic acid that is or is associated with the analyte). Section V provides kits according to the present disclosure, and Section VI describes exemplary applications of the present methods, compositions, and kits. As stated above, the section headings used herein are for organizational purposes only and are not to be construed as limiting the subject matter described.

### II. In Situ Targeted Shrinking and Expansion of Biomolecules

### A. Probes and Products Thereof

In some aspects, provided herein are methods and compositions for analyzing a biological sample comprising a target analyte, such as a biomolecule. The biological sample may comprise a probe or a product thereof (e.g., a nucleic acid concatemer, such as an RCP) that comprises one or more functional groups B. In some embodiments, the probe comprises a nucleic acid and is configured to directly or indirectly bind to the target analyte in the biological sample.

The methods and compositions disclosed herein can be used to detect and analyze a wide variety of different target analytes. In some embodiments, the target analyte can include any biological substance, structure, moiety, or component to be analyzed. Additional description of target analytes compatible with the methods provided herein can be found in Section IV.B below.

In some embodiments wherein the biological sample is embedded in a matrix (e.g., a hydrogel matrix), the target analyte may be attached to the matrix. In some embodiments, the target analyte comprises one or more functional groups for attachment to the matrix. In some embodiments, the one or more functional groups for attachment to the matrix are different from one or more functional groups that are used to attach the probes or products thereof (e.g., RCPs) to the polymer or polymer network (e.g., one or more functional groups A and/or one or more functional groups B). In some embodiments, the target analyte comprises one or more functional groups C for attachment to the matrix. In some embodiments, the target analyte is attached to the matrix via one or more functional groups C. In some embodiments, the one or more functional groups C comprise any of the functional groups described herein. In some embodiments, the one or more functional groups C are different from the one or more functional groups A and/or the one or more functional groups B. The target analyte comprising one or more functional groups C may be attached to the matrix using any of the reaction techniques described herein. In some embodiments, the probe or product thereof is attached to the matrix via hybridization with the target analyte. In some embodiments, the probe or product thereof may be directly attached matrix. In some embodiments, the probe or product thereof (e.g., RCP) is attached to the matrix via a functional group, such as any of the functional groups provided herein. In some embodiments, the probe or product thereof (e.g., RCP) comprising one or more functional groups may be attached to the matrix using any of the reaction techniques described herein. In some embodiments, the one or more functional groups (in the target analyte, or in the probe or product thereof (e.g., RCP)) for attachment to the matrix are used to maintain a relatively position of the target analyte or the probe or product thereof in the sample, whereas the reaction between functional groups A and B (in the polymer or polymer network and the probes or products thereof (e.g., RCPs), respectively) are used for selective shrinking or expansion of the probes or products thereof, e.g., without shrinking or expansion of the sample as a whole.

In some embodiments, the target analyte is a nucleic acid target analyte. In some embodiments, a probe comprising a nucleic acid binds the target analyte. In some embodiments, the probe is configured to directly bind to the target analyte in the biological sample. In some embodiments, the probe is configured to indirectly bind to the target analyte (e.g., via a labelling agent) in the biological sample.

The nucleic acid target analyte may be located in a DNA region that is inaccessible, or less accessible compared with other regions of DNA. In some embodiments, the nucleic acid target analyte is in chromatin region. In some embodiments, expansion of the probe or product thereof open the chromatin region. In some embodiments, the nucleic acid target analyte is in an open chromatin region. In some embodiments, expansion of the probe or product thereof opens the open chromatin region. In some embodiments, the nucleic acid target analyte is in a closed chromatin region. In some embodiments, expansion of the probe or product thereof opens the closed chromatin region. Opening of the chromatin region allows for improved resolution of the target nucleic acid analyte within the chromatin region, as the chromatin region becomes more accessible to detectable probes during downstream analysis.

A product of the target analyte can also be processed according to the methods provided herein. In some embodiments, the product of the target analyte is a product of a probe comprising a nucleic acid that directly or indirectly binds to the target analyte. For example, the product of the target analyte may be an amplification product, such as nucleic acid concatemer. In some embodiments, the probe is used as a primer for amplification of the target nucleic acid analyte. In some embodiments, the amplification product is an RCP. In some embodiments, the nucleic acid concatemer (e.g., RCP) comprises nucleic acids. In some embodiments, the nucleic acid concatemer contains natural and unnatural nucleotides. For example, the nucleic acid concatemer may comprise modified nucleotides, non-nucleotides, or synthetic nucleotides. Nucleotides amendable to the present application include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Additional suitable bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages. In some embodiments, the modified nucleotides are amine-modified nucleotides.

In some embodiments, the nucleic acid concatemer is between about 1 and about 85 kilobases in length, such as between any of about 1 and about 15 kilobases, about 10 and about 30 kilobases, about 20 and about 40 kilobases, about 30 and about 50 kilobases, about 40 and about 60 kilobases, about 50 and about 70 kilobases, and about 60 and about 85 kilobases in length. In some embodiments, the nucleic acid concatemer is at least 1 kilobase in length, such as any of about 15, 25, 35, 45, 55, 65, or 85 kilobases in length. In some embodiments, the nucleic acid concatemer is more than 85 kilobases in length.

In some embodiments, the nucleic acid concatemer is an RCP comprising a barcode sequence corresponding to an analyte in the biological sample, and the nucleic acid concatemer is between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length.

In some embodiments, the nucleic acid concatemer forms a nanoball such as one having a diameter between about 0.1 μm and about 3 μm. The nanoball may be a product of amplification, such as RCA. In some embodiments, the nanoball has a diameter of between about 0.1 μm and about 4 μm, such as between any of about 0.1 μm and about 0.5 μm, about 0.2 μm and about 2 μm, about 1 μm and about 3 μm, and about 2 μm and about 4 μm. In some embodiments, the nanoball diameter is at least about 0.1 μm, such as at least any of about 0.2 μm, 0.5 μm, 1 μm, 1.5 μm, 2 μm, 2.5 μm, 3 μm, 3.5 μm, and 4 μm.

In some embodiments, a nucleic acid concatemer is generated from a nucleic acid analyte. For example, the nucleic acid analyte may be DNA, ssDNA, or RNA. In some embodiments, the nucleic acid concatemer is generated from a labelling agent that directly or indirectly binds to an analyte in the biological sample. Examples of labelling agents are described in Section IV.B below.

In some embodiments, the nucleic acid concatemer is an amplification product, such as an RCP. RCA may comprise contacting the biological sample with one or more probes (e.g., a circular probe or circularizable probe or probe set) to produce an RCP. RCA, and RCPs thereof, is further described in Section IV.B below.

In some embodiments, the biological sample comprises a plurality of primary probes each hybridized to a target sequence in the target nucleic acid. In some embodiments, the plurality of primary probes each hybridizes to a different target sequence in the target nucleic acid. In some embodiments, the plurality of primary probes each hybridizes to the same target sequence in the target nucleic acid. In some embodiments, the primary probe comprises nucleic acids. In some embodiments, the plurality of primary probes comprises one or more functional groups B. In some embodiments, the product of the plurality of primary probes comprises one or more functional groups B. In some embodiments, a complex comprising a plurality of primary probes comprises one or more functional groups B. In some embodiments, the product of the primary probe is an amplification product. In some embodiments, the product of the primary probe is an RCP. In some embodiments, the complex of the primary probe is a complex comprising an initiator and an amplifier for hybridization chain reaction (HCR). In some embodiments, the complex of the primary probe is a complex comprising an initiator and an amplifier for linear oligonucleotide hybridization chain reaction (LO-HCR). In some embodiments, the product of the primary probe is a primer exchange reaction (PER) product. In some embodiments, the complex of the primary probe is a complex comprising a pre-amplifier and an amplifier for branched DNA (bDNA). In some embodiments, the product of the primary probe is an RCP and nucleotides comprising one or more functional groups B are incorporated into the RCP during RCA, e.g., the primary probe is used as a template for an RCA reaction such that the nucleotides comprising one or more functional groups B are incorporated into the RCP during RCA. In some embodiments, the RCP is shrunk.

In some embodiments, the primary probe comprises an overhang upon hybridization to the target sequence. In some embodiments, the overhang comprises one or more barcode sequences (e.g., sequences that permit identification of the primary probe and/or the target sequence which the primary probe hybridizes to). In some embodiments, each barcode of the one or more barcode sequences are different. In some embodiments, the primary probe comprises a 3′ overhang upon hybridization to the target sequence. In some embodiments, the 3′ overhang comprises one or more barcode sequences. In some embodiments, the primary probe comprises a 5′ overhang upon hybridization to the target sequence. In some embodiments, the 5′ overhang comprises one or more barcode sequences. In some embodiments, the primary probe comprises a 3′ overhang and a 5′ overhang upon hybridization to the target sequence. In some embodiments, the 3′ overhang and the 5′ overhang each independently comprises one or more barcode sequences. In some embodiments, the one or more barcode sequences of the 3′ overhang are the same as the one or more barcode sequences of the 5′ overhang. In some embodiments, the one or more barcode sequences of the 3′ overhang are different from the one or more barcode sequences of the 5′ overhang.

In some embodiments, the primary probe is a circular primary probe. In some embodiments, the primary probe a circularizable primary probe or probe set. In some embodiments, the primary probe is a primary probe or probe set comprising a split hybridization region configured to hybridize to a splint. In some embodiments, the split hybridization region comprises one or more barcode sequences. In some embodiments, the primary probe comprises a combination of any of the features of the primary probes provided herein (e.g., comprises a overhang, is a circular primary probe, etc.). In some embodiments, the primary probe is a linear primary probe. In some embodiments, each primary probe of a plurality of primary probes can each be hybridized to a target sequence in the target nucleic acid.

In some embodiments, the primary probe (e.g., the primary probe hybridized to a target sequence in the target nucleic acid) is capable of binding with an intermediate probe. In some embodiments, the biological sample comprises a plurality of intermediate probes that directly bind to the primary probe or a product or complex thereof. In some embodiments, the biological sample comprises a plurality of intermediate probes that indirectly bind to the primary probe or a product or complex thereof. In some embodiments, each of the plurality of intermediate probes or a product or complex thereof independently comprises one or more functional groups B. In some embodiments, the product of the intermediate probe is an amplification product. In some embodiments, the product of the intermediate probe is an RCP. In some embodiments, the complex of the intermediate probe is a complex comprising an initiator and an amplifier for hybridization chain reaction HCR. In some embodiments, the complex of the intermediate probe is a complex comprising an initiator and an amplifier for LO-HCR. In some embodiments, the product of the intermediate probe is a PER product. In some embodiments, the complex of the intermediate probe is a complex comprising a pre-amplifier and an amplifier for bDNA. In some embodiments, the product of the intermediate probe is an RCP and nucleotides comprising one or more functional groups B are incorporated into the RCP during RCA, e.g., the intermediate probe is used as a template for an RCA reaction such that the nucleotides comprising one or more functional groups B are incorporated into the RCP during RCA. In some embodiments, the RCP is shrunk.

In some embodiments, the intermediate probe comprises an overhang upon hybridization to a primary probe or a product or complex thereof. In some embodiments, the overhang comprises one or more barcode sequences (e.g., sequences that permit identification of the intermediate probe and/or the primary probe or a product or complex thereof). In some embodiments, each barcode of the one or more barcode sequences are different. In some embodiments, the intermediate probe comprises a 3′ overhang. In some embodiments, the 3′ overhang comprises one or more barcode sequences. In some embodiments, the intermediate probe comprises a 5′ overhang. In some embodiments, the 5′ overhang comprises one or more barcode sequences. In some embodiments, the intermediate probe comprises a 3′ overhang and a 5′ overhang upon hybridization to a primary probe or a product or complex thereof. In some embodiments, the 3′ overhang and the 5′ overhang each independently comprises one or more barcode sequences. In some embodiments, the one or more barcode sequences of the 3′ overhang are the same as the one or more barcode sequences of the 5′ overhang. In some embodiments, the one or more barcode sequences of the 3′ overhang are different from the one or more barcode sequences of the 5′ overhang.

In some embodiments, the target analyte is a non-nucleic acid analyte. In some embodiments, a probe comprising a nucleic acid binds the target analyte. In some embodiments, the probe is configured to directly bind to the target analyte in the biological sample. In some embodiments, the probe is configured to indirectly bind to the target analyte (e.g., via a labelling agent) in the biological sample. In some embodiments, the probe comprises a target-binding moiety and a reporter oligonucleotide. In some embodiments, the target-binding moiety directly binds to the non-nucleic acid target. In some embodiments, the target-binding moiety indirectly binds (e.g., via a labeling agent) to the non-nucleic acid target.

In some embodiments, the one or more functional groups B are in the target-binding moiety. In some embodiments, the one or more functional groups B are in the reporter oligonucleotide. In some embodiments, the one or more functional groups B are in the target-binding moiety and the reporter oligonucleotide. In some embodiments, the one or more functional groups B in the target-binding moiety and the reporter oligonucleotide are the same functional groups.

In some embodiments, the target-binding moiety is an antibody or epitope binding fragment thereof. In some embodiments, the target-binding moiety includes one or more antibodies or epitope binding fragments thereof. The antibodies or epitope binding fragments including the target-binding moiety can specifically bind to a target analyte. In some embodiments, the target analyte is a protein (e.g., a protein on a surface of the biological sample (e.g., a cell) or an intracellular protein). In some embodiments, a plurality of labeling agents comprising a plurality of binding moieties bind a plurality of target analytes present in a biological sample. In some embodiments, the plurality of target analytes includes a single species of target analyte (e.g., a single species of polypeptide). In some embodiments in which the plurality of target analytes includes a single species of target analyte, the binding moieties of the plurality of labeling agents are the same. In some embodiments in which the plurality of target analytes includes a single species of target analyte, the binding moieties of the plurality of labeling agents are the different (e.g., members of the plurality of target analyte labeling agents can have two or more species of binding moieties, wherein each of the two or more species of target analyte binding moieties binds a single species of target analyte, e.g., at different binding sites). In some embodiments, the plurality of target analytes includes multiple different species of target analyte (e.g., multiple different species of polypeptides).

In some embodiments, a nucleic acid target analyte or a non-nucleic acid target analyte can be shrunk using methods disclosed herein. In some embodiments, the shrinking reduces the space occupied by the nucleic acid or non-nucleic acid target analytes. In some embodiments, the shrinking expands the space between adjacent target analytes and/or probes or products thereof at different locations in the biological sample. For example, the shrinking expands the space between individual target analytes and/or probes or products thereof at neighboring locations in the biological sample. In some embodiments, the shrinking creates more space for analyzing additional biomolecules (multiplexing) simultaneously in the location in the biological sample. For example, the shrinking may achieve smaller and more compact signals associated with each analyte and better separate a signal associated with one analyte from a signal associated with another analyte.

In some embodiments, the probe or product thereof comprises a region that directly or indirectly binds to a compaction oligonucleotide. In some embodiments, the compaction oligonucleotide comprises two or more hybridization regions that hybridize to compaction regions in the probe or product thereof (e.g., RCP). In some embodiments, the compaction oligonucleotide comprises a spacer region between the two or more hybridization regions.

The probe or product thereof comprises one or more functional groups B (e.g., as shown in FIGS. 1-4). In some embodiments wherein the target analyte is a nucleic acid target analyte, the one or more functional groups B are in one or more nucleotides on the target analyte. In some embodiments, the one or more functional groups B are in a product of the probe directly or indirectly bound to the target analyte. For example, in some embodiments wherein the target analyte is a nucleic acid target analyte, the one or more functional groups B are in an amplification product (e.g., a nucleic acid concatemer, such as an RCP) of the probe directly or indirectly bound to the target analyte. In some embodiments, the one or more functional groups B are in one or more molecules of the probe bound to the target analyte. For example, in some embodiments wherein the target analyte is a non-nucleic acid target analyte, the one or more functional groups B are in one or more molecules of the probe (e.g., an antibody) bound to the non-nucleic acid target analyte (e.g., a protein).

In some embodiments, the probe or product thereof is pre-functionalized with one or more functional groups B. In some embodiments, the method further comprises functionalizing the probe or product thereof with functional groups B. In some embodiments, the probe is functionalized with one or more functional groups B prior to binding the target analyte in the biological sample. In some embodiments, the probe is functionalized with one or more functional groups B after binding the target analyte in the biological sample. In some embodiments, the one or more functional groups B are capable of reacting with one or more functional groups A of a polymer, thereby forming a polymer conjugate.

The probe or product thereof may be functionalized for downstream analysis (e.g., in situ detection such as by sequencing or sequential hybridization of detectable probes). In some embodiments, the probe or product thereof comprises a detectable label. The detectable label may be any of the detectable labels described herein and in Section III below. In some embodiments, the probe or product thereof comprises a region that directly binds to a detectably labeled probe (e.g., a probe comprises a detectable label, such as any of the detectable labels described herein). In some embodiments, the probe or product thereof comprises a region that indirectly binds to a detectably labeled probe.

### B. Polymer Conjugate and Polymer Network

A polymer conjugate may be formed by contacting the biological sample with a polymer. For example, the polymer may react with a probe or product thereof in the biological sample to form the polymer conjugate. Specifically, the polymer comprises a functional group A capable of reacting with a functional group B in the probe or product thereof in the biological sample to form the polymer conjugate. In some aspects, provided herein are methods and compositions for analyzing a biological sample comprising a polymer conjugate. In some embodiments, a polymer conjugate is formed upon reaction of one or more functional groups A of a polymer with one or more functional groups B of a probe or product thereof. In some embodiments, the reaction of one or more functional groups A with one or more functional groups B couples the polymer to the probe or product thereof to form the polymer conjugate. In some embodiments, the probe or product thereof is conjugated to one or more polymer molecules. In some embodiments, the probe or product thereof is conjugated to multiple polymer molecules. In some instances, the polymer comprises a synthetic artificial crosslinking system that can be used for selective crosslinking.

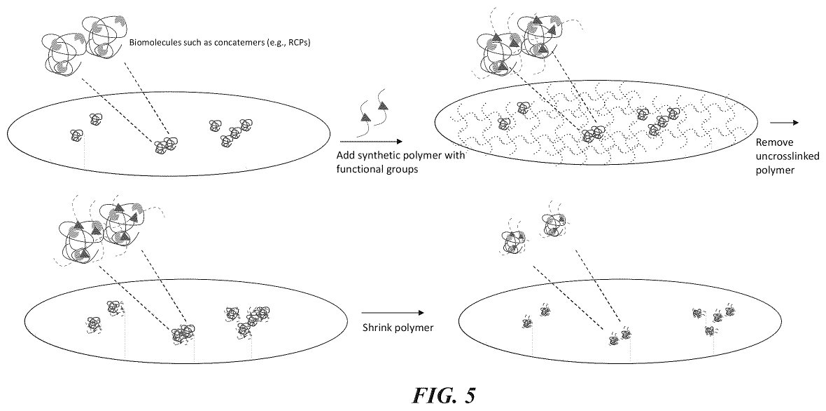

FIG. 5 shows an exemplary workflow for in situ shrinking of biomolecules in a biological tissue sample. Briefly, nucleic acid concatemers such as an RCPs are generated using nucleotides with functional groups. The biological sample is then contacted with polymer comprising one or more functional groups, which are reacted (e.g., crosslinked) to the functional groups on the nucleotides in the RCPs to form a polymer conjugate. Once the cross-linked polymer network is formed, unreacted (e.g., uncrosslinked) polymer in the tissue sample can be removed. The polymer conjugate is shrunk, thereby selectively shrinking the biomolecules maintaining its location in the tissue sample while other portions of the tissue sample remains the same size.

In some embodiments, the polymer is pre-functionalized with one or more functional groups A. In some embodiments, the method further comprises functionalizing the polymer with functional groups A. In some embodiments, the polymer is functionalized with one or more functional groups A prior to contacting the biological sample with the polymer.

In some aspects, the method further comprises processing or clearing the biological sample. Processing or clearing the biological sample may comprise, but is not limited to, techniques described in Section IV.A below. In some embodiments, contacting the biological sample with the polymer (e.g., polymer monomers or polymer network) occurs before processing or clearing the biological sample. In some embodiments, contacting the biological sample with the polymer occurs after processing or clearing the biological sample. In some embodiments, contacting the biological sample with the polymer occurs during processing or clearing the biological sample.

In some embodiments wherein the biological sample is embedded in a matrix (e.g., a hydrogel matrix), the polymer may be attached to the matrix. In some embodiments, the polymer comprises one or more functional groups D for attachment to the matrix. In some embodiments, the polymer is attached to the matrix via one or more functional groups D. In some embodiments, the one or more functional groups D may be any of the functional groups described herein. The polymer comprising one or more functional groups D may be attached to the matrix using any of the reaction techniques described herein. In some embodiments, functional group D may comprise a cationic species such as alkyl ammonium, quaternary pyridine, or imidazolium. In some embodiments, functional group D may comprise an anionic species such as phosphate, carboxylate, or sulfonate.

In some embodiments, functional group A may comprise a cationic species such as alkyl ammonium, quaternary pyridine, or imidazolium. In some embodiments, functional group A may comprise an anionic species such as phosphate, carboxylate, or sulfonate. In some embodiments, functional group B may comprise a cationic species such as alkyl ammonium, quaternary pyridine, or imidazolium. In some embodiments, functional group B may comprise an anionic species such as phosphate, carboxylate, or sulfonate.

In some embodiments, the reaction between functional group A and functional group B forms a covalent bond. In some embodiments, the reaction between functional group A and functional group B is a click reaction. In some such embodiments, the click reaction between functional group A and functional group B is biorthogonal. In some embodiments, the click reactions between functional groups A and functional groups B are orthogonal to one or more enzymatic reactions in the sample. In some embodiments, the functional groups that participate in the click reaction (e.g., functional group A and functional group B) may comprise, but are not limited to, azido/alkynyl; azido/cyclooctynyl; tetrazine/dienophile; thiol/alkynyl; cyano/1,2-amino thiol; and, nitrone/cyclooctynyl.

In some embodiments, functional group A is azido and functional group B is alkynyl. In some embodiments, functional group A is alkynyl and functional group B is azido. In some embodiments, functional group A is azido and functional group B is cyclooctynyl. In some embodiments, functional group A is cyclooctynyl and functional group B is azido. In some embodiments, functional group A is a tetrazine and functional group B is a dienophile. In some embodiments, functional group A is a dienophile and functional group B is a tetrazine. In some embodiments, functional group A is a thiol and functional group B is alkynyl. In some embodiments, functional group A is alkynyl and functional group B is a thiol. In some embodiments, functional group A is cyano and functional group B is a 1,2-aminothiol. In some embodiments, functional group A is a 1,2-aminothiol and functional group B is cyano. In some embodiments, functional group A is a nitrone and functional group B is cyclooctynyl. In some embodiments, functional group A is cyclooctynyl and functional group B is a nitrone.

In some embodiments, the polymer is a synthetic polymer, a natural polymer, or combination thereof. In some embodiments, the polymer is a synthetic polymer. In some embodiments, the polymer is a natural polymer. In some embodiments, the polymer is a combination or copolymer of a synthetic polymer and a natural polymer. In some embodiments, the polymer is a homopolymer. In some embodiments, the polymer is a synthetic homopolymer. In some embodiments, the polymer is a natural homopolymer. In some embodiments, the polymer is a copolymer of two or more synthetic polymers. In some embodiments, the polymer is a copolymer of two or more natural polymers. In some embodiments, the polymer is a copolymer of one or more synthetic polymers and one or more natural polymers. In some embodiments, the polymer is a statistical copolymer. In some embodiments, the polymer is a random copolymer. In some embodiments, the polymer is a block copolymer.

In some embodiments, the polymer is bifunctional. In some embodiments, the polymer comprises one or more functional groups A per molecule of polymer. In some embodiments, the polymer may comprises functional groups in addition to the one or more functional groups A In some embodiments, the polymer comprises one or more spacers between functional groups A. In some embodiments, the spacer comprises at least one group selected from C1-6 alkylene, C2-6 alkenylene, C2-6 alkynylene, C═O, O, S, NH, —(C═O)O—, —(C═O)NH—, —S—S—, ethylene glycol, polyethyleneglycol (PEG), propylene glycol, and polypropyleneglycol, or any combination thereof.

In some embodiments, the polymer is selected from the group consisting of poly(acrylate), poly(alkyl-acrylate), poly(hydroxyalkyl-acrylate), poly(acrylamide), poly(alkyl-acrylamide), poly(ethylene glycol), poly(vinyl alcohol), poly(vinyl pyrrolidone), poly(lactic acid), poly(lactic-co-glycolic acid), hyaluronic acid, chitosan, heparin, alginate, fibrin, collagen, gelatin, and copolymers of any two or more of the foregoing. In some embodiments, the polymer is a poly(acrylate). In some embodiments, the polymer is a poly(alkyl-acrylate). In some embodiments, the polymer is a poly(hydroxyalkyl-acrylate). In some embodiments, the polymer is a poly(acrylamide). In some embodiments, the polymer is a poly(alkyl-acrylamide). In some embodiments, the polymer is a poly(ethylene glycol). In some embodiments, the polymer is a poly(vinyl alcohol). In some embodiments, the polymer is a poly(vinyl pyrrolidone). In some embodiments, the polymer is a poly(lactic acid). In some embodiments, the polymer is a poly(lactic-co-glycolic acid). In some embodiments, the polymer is hyaluronic acid. In some embodiments, the polymer is chitosan. In some embodiments, the polymer is heparin. In some embodiments, the polymer is alginate. In some embodiments, the polymer is fibrin. In some embodiments, the polymer is collagen. In some embodiments, the polymer is gelatin.

In some embodiments, unreacted polymer is removed from the biological sample. In some embodiments, the removing comprises washing the biological sample. In some embodiments, the biological sample is washed with a washing solution. The washing solution may comprise any suitable reagent for removing unreacted polymers from the biological sample. In some embodiments, the removing unreacted polymer does not disrupt the polymer conjugate. In some embodiments, the removing unreacted polymer does not disrupt the polymer network. In some embodiments, the removing unreacted polymer does not disrupt the location of (e.g., does not move) the probe or product thereof in the biological sample. In some embodiments, the removing unreacted polymer does not disrupt the location of (e.g., does not move) the target analyte in the biological sample.

In some embodiments, the polymer comprises polymerizable monomers or polymers, or cross-linkable polymers. The polymer may form a polymer network. In some embodiments, the polymer network is formed upon cross-linking one or more polymers. In some embodiments, the polymer network is formed prior to contacting the biological sample with the polymer (FIGS. 1 and 3). For example, the biological sample may be contacted with a pre-cross-linked polymer network comprising one or more functional groups A. In some embodiments, the polymer network is formed prior to forming the polymer conjugate in the biological sample. For example, the biological sample may be contacted with a plurality of polymers, polymer-polymer cross-linking is initiated to form the polymer network between the polymers within the biological sample, and the polymer network subsequently reacts with the probe or product thereof in the biological sample. In some embodiments, the polymer network is formed upon cross-linking one or more polymers present in a polymer conjugate (FIGS. 2 and 4).

In some embodiments, the method comprises initiating polymer-polymer cross-linking to form a polymer network between the polymers (e.g., polymers of the polymer conjugate or unreacted polymers). In some embodiments, the reaction between one or more functional groups A on the polymer and one or more functional groups B on the probe or product thereof forms a polymer network between the polymers of the polymer conjugate. In some embodiments, the polymer exists as a polymer network prior to the reaction of the one or more functional groups A and the one or more functional groups B. In other embodiments, the polymer network is formed by initiating polymer-polymer cross-linking subsequent to reaction of one or more functional groups A and the one or more functional groups B. In some embodiments, the polymer network is formed by the reaction of one or more functional groups A and the one or more functional groups B.

In some embodiments, the polymer-polymer cross-linking creates a physically cross-linked polymer network. In some embodiments, the physical cross-linking occurs through ion-ion interactions between the polymers. In some embodiments, the physical cross-linking occurs through hydrogen bonding between the polymers. In some embodiments, the physical cross-linking occurs through forming a covalent between the polymers.

Cross-linking can also be performed chemically and/or photochemically. In some embodiments, the polymer-polymer cross-linking creates a chemically cross-linked polymer network. In some embodiments, the chemically cross-linked polymer network is created through photo-crosslinking of the polymers. In some embodiments, the method further comprises adding a photoinitiator. In some embodiments, the photoinitiatior initiates the photo-crosslinking of the polymers. In some embodiments, the photoinitatior is selected from the group consisting of: benzoyl peroxide, 2,2-dimethoxy-2-phenylacetophenone, 2-hydroxy-2-methyl-1-phenylpropanone, 1-hydroxy-cyclohexylphenylketone, benzophenone, isopropyl thioxanthone, and an organic peroxide. In some embodiments, the chemically cross-linked polymer network is created by reacting the polymers with a cross-linking reagent. In some embodiments, the cross-linking reagent is a multivalent (e.g., bivalent or trivalent) reagent comprising functional groups selected from the group consisting of a thiol, epoxide, amine, alcohol, hydrazide, hydroxylamine, and isocyanate.

In some embodiments, the chemically cross-linked polymer network is created by a click reaction between the polymers. In some such embodiments, the click reaction between the polymers biorthogonal. In some embodiments, the click reaction between the polymers is orthogonal to one or more enzymatic reactions in the sample. In some embodiments, the click reaction between the polymers is orthogonal to the click reaction between functional group A of the polymer and functional group B of the probe or product thereof.

In some embodiments, the functional groups that participate in the click reaction between the polymers may comprise, but are not limited to, azido/alkynyl; azido/cyclooctynyl; tetrazine/dienophile; thiol/alkynyl; cyano/1,2-amino thiol; and, nitrone/cyclooctynyl. In some embodiments, a first functional group is azido and a second functional group is alkynyl. In some embodiments, a first functional group is alkynyl and a second functional group is azido. In some embodiments, a first functional group is azido and a second functional group is cyclooctynyl. In some embodiments, a first functional group is cyclooctynyl and a second functional group is azido. In some embodiments, a first functional group is a tetrazine and a second functional group is a dienophile. In some embodiments, a first functional group is a dienophile and a second functional group is a tetrazine. In some embodiments, a first functional group is a thiol and a second functional group is alkynyl. In some embodiments, a first functional group is alkynyl and a second functional group is a thiol. In some embodiments, a first functional group is cyano and a second functional group is a 1,2-aminothiol. In some embodiments, a first functional group is a 1,2-aminothiol and a second functional group is cyano. In some embodiments, a first functional group is a nitrone and a second functional group is cyclooctynyl. In some embodiments, a first functional group is cyclooctynyl and a second functional group is a nitrone.

In some embodiments, the polymer network is porous, thereby permitting the introduction of reagents into the polymer network at the site of the target analyte (e.g., a probe or product thereof that is bound to the target analyte). To make a porous polymer, a suitable ratios of polymers to probes or products thereof may be selected to control the cross-linking density. In some embodiments, control over the molecular sieve size and density is achieved by adding additional cross-linkers such as functionalized polyethylene glycols.

In some embodiments, the polymer network is sufficiently optically transparent and/or may have optical properties compatible with the detection and analysis techniques described herein (see, e.g., Section III below), such as standard sequencing chemistries and/or imaging.

C. Shrinking and/or Expanding the Polymer Conjugate

The polymer conjugate may be shrunk or expanded in the biological sample. In some embodiments, shrinking or expanding the polymer conjugate respectively shrinks or expands the probe or product thereof. In some embodiments, the polymer conjugate is shrunk. In some embodiments, the probe or product thereof is shrunk. In some embodiments, the polymer conjugate is expanded. In some embodiments, the probe or product thereof is expanded. As the probe is capable of binding a target analyte, shrinking or expanding the probe or product thereof allows for increased resolution of the target analyte during downstream analysis. In some cases, the shrinking or expanding of the polymer is activatable. In some embodiments, shrinking the probe or product thereof allows for increased resolution or signal intensity during the detection. In some embodiments, expanding the probe or product thereof allows for increased accessibility to the biomolecule (e.g., a concatemer such as a RCP). In some cases, the increased accessibility from expanding allows for increased binding of detectably labeled probes to the biomolecule for detection. In some cases, the increased accessibility from expanding allows for increased removal of detectably labeled probes from the biomolecule after detection.

In some embodiments, the crosslinking of the probes or products thereof to the polymer is performed in two or more steps. In some instances, the polymer is first crosslinked to the targeted functional groups (e.g., on the nucleotides of the probes or products thereof) and then the sample is washed to remove uncrosslinked polymer. In some instances, then the polymers can be crosslinked with each other, for example using a second crosslinking chemistry (e.g., different functional groups) that is independent of the chemistry used to crosslink the polymer to the probes or products thereof. In some embodiments, the polymers of the second crosslinking chemistry is used for controlled shrinkage.

In some embodiments, shrinking or expanding the polymer conjugate is initiated by contacting the biological sample with a solution. In some embodiments, the solution is selected from the group consisting of: water, aqueous inorganic acid, aqueous organic acid, aqueous inorganic base, aqueous organic base, aqueous organic salt solution, and aqueous inorganic salt solution. In some embodiments, contacting the biological sample with a solution is performed prior to detection (e.g., sequencing and/or imaging). In some embodiments, the solution is removed from the biological sample prior to detection. In some embodiments, contacting the biological sample with a solution is performed after detection. In some embodiments, contacting the biological sample with a solution is performed prior to detection and the biological sample remains in contact with the solution during detection.

In some examples, the buffer composition for polymer conjugate or network shrinking comprises a majority of water. For example, compared to the buffer composition that allows the polymer conjugate or network to expand (or compared to a buffer composition that does not promote or facilitate expansion/loosening or shrinking of the polymer conjugate or network), the buffer that promotes polymer conjugate or network shrinking comprises a higher water content. In some embodiments, the buffer that promotes polymer conjugate or network shrinking comprises at least or about 50% H₂O, at least or about 55% H₂O, at least or about 60% H₂O, at least or about 65% H₂O, at least or about 70% H₂O, at least or about 75% H₂O, at least or about 80% H₂O, at least or about 85% H₂O, at least or about 90% H₂O, or at least or about 95% H₂O.

In some examples, the buffer composition for polymer conjugate or network expanding comprises a high DMSO content. For example, compared to the buffer composition that allows the polymer conjugate or network to shrink (or compared to a buffer composition that does not promote or facilitate expansion/loosening or shrinking of the polymer conjugate or network), the buffer that promotes polymer conjugate or network expanding or loosening comprises a higher DMSO concentration. In some embodiments, the buffer composition for polymer conjugate or network expanding or loosening comprises DMSO, H₂O, and a detergent or surfactant. In some embodiments, the buffer composition for polymer conjugate or network expanding or loosening comprises DMSO, H₂O, and a salt. In some embodiments, the buffer composition for polymer conjugate or network expanding or loosening comprises DMSO, H₂O, a detergent or surfactant, and a salt. In some embodiments, the buffer composition for polymer conjugate or network expanding or loosening comprises at least or about 25% DMSO, at least or about 30% DMSO, at least or about 35% DMSO, at least or about 40% DMSO, at least or about 45% DMSO, at least or about 50% DMSO, at least or about 55% DMSO, at least or about 60% DMSO, at least or about 65% DMSO, at least or about 70% DMSO, or at least or about 75% DMSO. In some embodiments, the buffer composition for polymer conjugate or network expanding or loosening comprises about 0.01%, about 0.05%, about 0.1%, about 0.5%, about 1%, or about 5% of a detergent or surfactant, such as a polysorbate-type nonionic surfactant, e.g., one that is formed by the ethoxylation of sorbitan monolaurate, e.g., Tween™ 20. In some embodiments, the buffer composition for polymer conjugate or network expanding or loosening comprises no more than or about 75% H₂O, no more than or about 70% H₂O, no more than or about 65% H₂O, no more than or about 60% H₂O, no more than or about 55% H₂O, no more than or about 50% H₂O, no more than or about 45% H₂O, no more than or about 40% H₂O, no more than or about 35% H₂O, no more than or about 30% H₂O, or no more than or about 25% H₂O. In some embodiments, the buffer composition for polymer conjugate or network expanding or loosening comprises about 0.1%, about 0.5%, about 1%, about 1.5%, about 2%, about 2.5%, about 3%, about 3.5%, about 4%, about 4.5%, or about 5% of a salt, such as KCl. In some embodiments, the buffer composition for polymer conjugate or network expanding or loosening comprises 50% DMSO, 0.1% Tween™ 20, 47.5% H₂O, and 2.6% KCl. In some embodiments, the buffer composition for polymer conjugate or network expanding facilitates probe removal (e.g., stripping detectably labeled probes).

In some embodiments, shrinking or expanding the polymer conjugate is initiated by changing the temperature of the biological sample. In some embodiments, the temperature of the biological sample is increased. In some embodiments, the temperature is decreased. In some embodiments, the temperature of the biological sample is increased. In some embodiments, changing the temperature of the biological sample is performed prior to detection (e.g., sequencing and/or imaging). In some embodiments, changing the temperature of the biological sample is performed during detection. In some embodiments, changing the temperature of the biological sample is performed prior to detection and remains changed during detection.

In some embodiments, the polymer is photosensitive or comprises a photosensitive functional group. In some embodiments wherein the polymer is photosensitive or comprises a photosensitive functional group, shrinking or expanding the polymer conjugate is initiated by exposing the biological sample to light. In some embodiments, exposing the biological sample to light is performed prior to detection (e.g., sequencing and/or imaging). In some embodiments, exposing the biological sample to light is performed during detection. In some embodiments, exposing the biological sample to light is performed prior to detection and continues during detection. In some embodiments, exposing the biological sample to light is performed prior to imaging. In some embodiments, exposing the biological sample to light is performed during imaging. In some embodiments, exposing the biological sample to light is performed prior to imaging and continues during imaging. In some embodiments, the light is used to image the biological sample and/or detect a signal associated with the probe or product thereof or the target analyte. In some embodiments, the light used to initiate the shrinking or expanding is the same light that is used to image the biological sample and/or detect a signal associated with the probe or product thereof or the target analyte. In some embodiments, shrinking or expanding the polymer conjugate occurs during the imaging.

In some embodiments, the polymer, polymer network, and/or polymer conjugate is degradable, and the method can comprise degrading the polymer conjugate. In some cases, some portion of the polymer in the sample is degraded and removed from the sample. The polymer conjugate may be degraded without disrupting the biological sample. For example, in some aspects, the polymers of the polymer conjugate are degraded. In some embodiments, the polymer network is degraded. In some embodiments, the polymer conjugate is degraded without disrupting (e.g., degrading) the polymer network. In some embodiments, the polymer conjugate is degraded without disrupting the probe or product thereof (e.g., RCP) in the biological sample. In some embodiments, the polymer conjugate is degraded without disrupting the location of the probe or product thereof in the biological sample. In some embodiments, the polymer conjugate is degraded without disrupting the target analyte. In some embodiments, the polymer conjugate is degraded without disrupting the location of the target analyte in the biological sample.

In some embodiments, disclosed herein is a degradable system where the polymer, polymer network, and/or polymer conjugate disclosed herein can be broken down or degraded by an external stimulus, thereby removing the polymer or polymer network from a biomolecule (e.g., a probe or product thereof such as RCPs) conjugated thereto and/or from the sample. In some embodiments, the polymer or polymer network can be removed from the sample, e.g., at one or more steps of stripping detectable probes from the sample. In some embodiments, a reagent may be added to a stripping buffer for use to strip detectable probes and to remove the polymer or polymer network (or a portion thereof). In some embodiments, the stripping buffer may comprise a reducing agent such as dithiothreitol (DTT) which can be used for the cleavage of disulfide bonds directly or indirectly linking the polymer or polymer network to the biomolecule. In some embodiments, the polymer, polymer network, and/or polymer conjugate is thermally degradable or cleavable from the biomolecule. In some embodiments, the polymer, polymer network, and/or polymer conjugate is optically degradable or cleavable from the biomolecule. In some embodiments, the polymer, polymer network, and/or polymer conjugate comprises a photodegradable and/or photocleavable moiety. In some embodiments, the polymer, polymer network, and/or polymer conjugate is linked to the biomolecule via a photodegradable and/or photocleavable linker. In some embodiments, the polymer, polymer network, and/or polymer conjugate is degradable and/or cleavable with a particular wavelength. In some embodiments, photo-degradation and/or photo-cleavage of the polymer, polymer network, and/or polymer conjugate can be used as an imaging based control, and the sizes of the polymer conjugate before and after photo-degradation and/or photo-cleavage can be compared.

In some embodiments, the degrading comprises contacting the biological sample with a stripping buffer. In some embodiments, the stripping buffer comprises a reducing agent, such as dithiothreitol (DTT). In some embodiments, the degrading comprises changing the temperature of the biological sample. In some embodiments, the degrading comprises increasing the temperature of the biological sample. In some embodiments, the degrading comprises decreasing the temperature of the biological sample. In some embodiments, the degrading comprises exposing the biological sample to light. In some embodiments, the wavelength of light is different from the wavelength of light used to detect a signal associated with the probe or product thereof or the target analyte in the biological sample.

In some embodiments, the degrading is performed before or during detection of a signal associated with the probe or product thereof or the target analyte in the biological sample. In some embodiments, the degrading is performed after detecting a signal associated with the probe or product thereof or the target analyte in the biological sample. In some embodiments, the degrading is performed simultaneously with removing a detectable probe from the probe or product thereof or the target analyte. In some embodiments, the method comprising further processing the biological sample, such as processing a biological sample according to any of the methods described herein (e.g., contacting the biological sample with a polymer, reacting the polymer with a probe or product thereof thereby forming a polymer conjugate, removing unreacted polymer from the biological sample, and shrinking or expanding the polymer conjugate), following degrading the polymer conjugate. For example, the biological sample may be contacted with another polymer and another polymer conjugate may be formed, and shrunk or expanded, following degradation of the polymer conjugate. In some embodiments, the method comprises contacting the biological sample with one or more detectable probes after further processing the biological sample according to any of the methods described herein (e.g., contacting the biological sample with a polymer, reacting the polymer with a probe or product thereof thereby forming a polymer conjugate, removing unreacted polymer from the biological sample, and shrinking or expanding the polymer conjugate). In some embodiments, the one or more detectable probes directly or indirectly bind to the probe or product thereof or the target analyte. In some embodiments, the method further comprises detecting signal(s) associated with the one or more detectable probes in the biological sample.

In some embodiments, the polymer conjugate (e.g., a polymer network with polymer conjugates comprising probes or probe products coupled to the polymer) may shrink and expand in a reversible manner. In some embodiments, repeated shrinking or expanding of the polymer conjugate (e.g., polymer conjugates in a polymer network) is initiated and performed by switching between different conditions which control the polymer conjugate. For example, repeated shrinking or expanding of the polymer conjugate (e.g., polymer conjugates in a polymer network) is performed by contacting the biological sample with different solutions (e.g., buffer compositions). In some embodiments, shrinking or expanding of the polymer conjugate (e.g., polymer conjugates in a polymer network) is performed by switching between two different buffers. In some embodiments, repeated shrinking or expanding of the polymer conjugate is activatable by changing the temperature of the biological sample or by using a photosensitive functional group.

In some embodiments, exposing the biological sample to a condition (e.g., buffer) that allows the polymer conjugate or polymer network formed by polymer conjugates to shrink is performed prior to detection (e.g., imaging). In some embodiments, exposing the biological sample to a buffer that allows the polymer conjugate or polymer network formed by polymer conjugates to shrink is performed during detection. In some embodiments, exposing the biological sample to a buffer that allows the polymer conjugate or polymer network formed by polymer conjugates to shrink is performed prior to detection and continues during detection. In some embodiments, exposing the biological sample to a buffer that allows the polymer conjugate or polymer network formed by polymer conjugates to shrink is performed prior to imaging. In some embodiments, exposing the biological sample to a buffer that allows the polymer conjugate or polymer network formed by polymer conjugates to shrink is performed during imaging. In some embodiments, exposing the biological sample to a buffer that allows the polymer conjugate or polymer network formed by polymer conjugates to shrink is performed prior to imaging and continues during imaging. In some embodiments, a buffer used during imaging maintains the shrunk polymer conjugate or polymer network. In some embodiments, the buffer that maintains the shrunk polymer conjugate or network comprises a phenolic acid, such as a dihydroxybenzoic acid. In some embodiments, the buffer that maintains the shrunk polymer conjugate or network comprises protocatechuic acid (PCA). In some embodiments, the buffer that maintains the shrunk polymer conjugate or network comprises a non-zwitterionic buffer. In some embodiments, the buffer that maintains the shrunk polymer conjugate or network comprises 1,3-bis(tris(hydroxymethyl)methylamino)propane, also known as BTP or BIS-TRIS propane. In some embodiments, the buffer comprises a dihydroxybenzoic acid and a non-zwitterionic buffer, such as PCA and BIS-TRIS propane, respectively, and the content and ratio of the dihydroxybenzoic acid (e.g., PCA) and non-zwitterionic buffer (e.g., BIS-TRIS propane) can be tuned to adjust the state of shrinking or loosening up of the polymer conjugate or network. In some examples, the buffer composition for shrinking the polymer conjugate or polymer network formed by polymer conjugates comprises a majority of water. For example, compared to the buffer composition that allows the polymer conjugate or polymer network formed by polymer conjugates to expand, the buffer that promotes shrinking of the polymer conjugate or polymer network formed by polymer conjugates comprises higher water content. In some instances, the signal associated with each of the target analytes (e.g., distance between probes or products thereof) should be maintained or not significantly changed compared to prior the shrinking to allow decoding and image analysis (e.g., decoding). In some aspects, shrinking the polymer conjugate or polymer network formed by polymer conjugates reduces the spread of the nanoball size compared to prior to shrinking the polymer conjugate or polymer network formed by polymer conjugates (e.g., in the range of 1-100 nanometers). In some aspects, while the conjugated probes or products thereof shrink, the biological sample and/or a matrix (e.g., a hydrogel matrix) embedding the biological sample or biomolecules therefrom as a whole maintains its dimension.

In some embodiments, exposing the biological sample to a condition (e.g., buffer) that allows the polymer conjugate or network to expand (or become less compact by “loosening”) is performed prior to hybridizing one or more detectably labeled probes to the probe or product thereof or the target analyte. In some embodiments, exposing the biological sample to a buffer that allows the polymer conjugate or network to expand is performed during hybridization of the one or more detectably labeled probes to the probe or product thereof or the target analyte. In some embodiments, exposing the biological sample to a buffer that allows the polymer conjugate or network to expand is initiated prior to hybridizing one or more detectably labeled probes to the probe or product thereof or the target analyte and continues during the hybridization step. In some examples, the buffer composition for polymer conjugate or network expanding comprises high DMSO. For example, compared to the buffer composition that allows the polymer conjugate or network to shrink, the buffer that promotes polymer conjugate or network loosening comprises higher DMSO concentration. In some aspects, expanding the polymer conjugate or network during the hybridizing of the one or more detectably labeled probes to the probe or product thereof or the target analyte may allow increased access of the detectably labeled probes to its targets (e.g., sequences in the amplification products (e.g., nanoball)). In some cases, the hybridization buffer used for hybridizing one or more detectably labeled probes initiates and maintains loosening of the polymer.

In any of the embodiments described herein, the shrinking and expanding or loosening up of the polymer network can be controlled by the selection of the monomers, tuning the ratio of two or more polymer monomer species (e.g., described in Section II.B.), and the mixing and activation of the functional groups used.

### D. Exemplary Embodiments of Shrinking the Polymer Conjugate

The method may comprise shrinking the polymer conjugate in the biological sample. Implosion fabrication is an example of shrinking polymer conjugate. See, e.g., U.S. Pat. No. 11,214,661, the contents of which is herein incorporated by reference in its entirety. Implosion fabrication involves adding a polymer network (e.g., polyacrylate) to a biological sample, crosslinking the polymer network to the biological sample, and shrinking the polymer network, thereby drawing all biomolecules within the sample closer together. Techniques such as implosion fabrication may be useful when applied to the context of shrinking nucleic acid concatemers to improve in situ detection. In contrast to some embodiments of the present disclosure, implosion fabrication is not a targeted approach, and therefore all biomolecules with a biological sample are shrunk.

In some embodiments, the method comprises shrinking a probe or product thereof in the biological sample, wherein the probe comprises a nucleic acid and is capable of binding with a target analyte in the biological sample. In some embodiments, the method comprises shrinking the target analyte in the biological sample.

Methods comprising shrinking the probe(s) or product thereof may be useful in embodiments where the target analyte is a nucleic acid target analyte. For example, the nucleic acid target analyte may be in an inaccessible region of DNA (e.g., a chromatin region). In other examples, the nucleic acid target analyte may be in a product of the nucleic acid target analyte, such as a nucleic acid concatemer. It can be advantageous to shrink nucleic acid concatemers for improved separation of individual concatemers, increased resolution during detection, increased multiplexing capabilities, and/or improved stability of the nucleic acid concatemers.

In some embodiments, the nucleic acid target analyte is in a product of a probe comprising one or more functional groups B. In some embodiments, the nucleic acid target analyte is in a product of a probe set, wherein at least one probe of the probe set comprises one or more functional groups B. For example, the probe may be used as a template in an amplification reaction to generate the product. In some embodiments, the product of the probe comprising one or more functional groups B is a nucleic acid product. In some embodiments wherein the product is a nucleic acid product, the polymer conjugate is a polymer conjugate. In some embodiments, the product is a nucleic acid concatemer. In some embodiments, contacting the biological sample with the polymer occurs before generation of the nucleic acid product in the biological sample. In some embodiments, contacting the biological sample with the polymer occurs during the generation of the nucleic acid product in the biological sample. In some embodiments, contacting the biological sample with the polymer occurs after generation of the nucleic acid product in the biological sample. In some embodiments, the nucleic acid product is generated in situ in the biological sample. In some embodiments, the nucleic acid product is immobilized in the biological sample.

In some embodiments, the nucleic acid product is a product of an endogenous nucleic acid molecule in the biological sample, such as any of the products of endogenous molecules described in Section IV.B.iii below. For example, the product may be an extension product, a replication product, a reverse transcription product, and/or an RCP. The endogenous nucleic acid molecules can be any of the endogenous nucleic acid molecules described in Section IV.B.i below, such as but not limited to a viral or cellular DNA or RNA, and a genomic DNA/RNA, mRNA, or cDNA. In some embodiments, the product is a product of a labelling agent that directly or indirectly binds to the target analyte in the biological sample. In some embodiments, the product is an extension product, a replication product, a reverse transcription product, an RCP, or a combination thereof. In some embodiments, the labelling agent comprises a reporter oligonucleotide. In some embodiments, the reporter oligonucleotide comprises one or more barcode sequences and the nucleic acid product comprises one or a plurality of copies of the one or more barcode sequences.

In some embodiments, the nucleic acid product is an RCP of a circular or circularizable probe or probe set that hybridizes to a DNA or RNA molecule in the biological sample. In some embodiments, the nucleic acid products generated from a plurality of different mRNA and/or cDNA molecules are analyzed, a barcode sequence in a particular circular or circularizable probe or probe set uniquely corresponds to a particular mRNA or cDNA molecule, and the particular circular or circularizable probe or probe set further comprises an anchor sequence that is common among circular or circularizable probes or probe sets for a subset of the plurality of different mRNA and/or cDNA molecules.

In some embodiments, the nucleic acid product is in the form of a nanoball. In some embodiments, shrinking the polymer conjugate reduces the spread of the nanoball size compared to the spread of the nanoball size prior to shrinking the polymer conjugate. In some embodiments, shrinking the polymer conjugate reduces the space occupied in the biological sample by the nanoball compared to the space occupied by the nanoball prior to shrinking the polymer conjugate. In some embodiments, shrinking the polymer conjugate reduces the diameter of the nanoball. For example, in some embodiments, the nanoball has a diameter of between about 0.1 μm and about 3 μm prior to shrinking the polymer conjugate, such as between any of about 0.1 μm and about 0.5 μm, between about 0.5 μm and about 1 μm, between about 0.8 μm and about 1.3 μm, or between about 1 μm and about 1.5 μm, prior to shrinking the polymer conjugate. Following shrinking of the polymer conjugate, in some embodiments, the nanoball has a diameter between about 0.1-fold and about 20-fold smaller, such as between any of about 0.1-fold and about 1-fold, between about 0.5-fold and about 5-fold, between about 2.5-fold and about 10-fold, between about 8-fold and about 15-fold, or between about 12-fold and about 20-fold smaller compared to the diameter prior to shrinking the polymer conjugate. In some embodiments, following shrinking of the polymer conjugate the nanoball has less than an about 20-fold smaller diameter compared to the diameter prior to shrinking the polymer conjugate, such as less than any of about 15-fold, 10-fold, 5-fold, 2.5-fold, 1-fold, 0.5-fold, 0.1-fold, or less, diameter compared to the diameter prior to shrinking the polymer conjugate. In some embodiments, following shrinking of the polymer conjugate the nanoball has greater than an about 0.1-fold smaller diameter compared to the diameter prior to shrinking the polymer conjugate, such as greater than any of about 0.5-fold, 1-fold, 2.5-fold, 5-fold, 10-fold, 15-fold, 20-fold, or greater, diameter compared to the diameter prior to shrinking the polymer conjugate.

In some embodiments, shrinking the polymer conjugate reduces the size of the nanoball and may allow for a reduction in amplification time for generating the product of the probe to be detected. For example, if the amplifying is achieved by performing rolling circle amplification (RCA), the RCA can be performed for no more than 15 minutes, for no more than 30 minutes, for no more than 45 minutes or for no more than 1 hour.

In some embodiments, provided herein is a method for processing a biological sample, comprising: (a) contacting the biological sample with a polymer, wherein the polymer comprises one or more functional groups A; the biological sample comprises a probe or a product thereof that comprises one or more functional groups B; and the probe comprises a nucleic acid and is configured to directly or indirectly bind to a nucleic acid target analyte in the biological sample; (b) reacting the one or more functional groups A with the one or more functional groups B, thereby coupling the polymer to the probe or product thereof to form a polymer conjugate; (c) removing unreacted polymer from the biological sample; and, (d) shrinking the polymer conjugate, thereby shrinking the probe or product thereof. In some embodiments, there is provided a method for processing a biological sample, comprising: (a) contacting the biological sample with a polymer, wherein: the polymer comprises one or more functional groups A; the biological sample comprises a product of a probe that comprises one or more functional groups B; the probe comprises a nucleic acid and is configured to directly or indirectly bind to a nucleic acid target analyte in the biological sample; and the product is a nucleic acid concatemer; (b) reacting the one or more functional groups A with the one or more functional groups B, thereby coupling the polymer to the probe or product thereof to form a polymer conjugate; (c) removing unreacted polymer from the biological sample; and, (d) shrinking the polymer conjugate, thereby shrinking the nucleic acid concatemer. In some embodiments, the nucleic acid concatemer is an RCP.

In some embodiments, provided herein is a method for processing a biological sample, comprising: (a) contacting the biological sample with a polymer, wherein the polymer comprises one or more functional groups A; the biological sample comprises a plurality of probes that comprises one or more functional groups B; and the plurality of probes directly or indirectly bind to a nucleic acid target analyte in the biological sample; (b) reacting the one or more functional groups A with the one or more functional groups B, thereby coupling the polymer to the plurality of probes to form a polymer conjugate; (c) removing unreacted polymer from the biological sample; and, (d) shrinking the polymer conjugate, thereby shrinking the plurality of probes. In some embodiments, the plurality of probes are used for fluorescent in situ hybridization based detection of one or more analytes, where the plurality of probes may but do not need to extended or amplified using a polymerase. In some instances, the plurality of probes comprises a plurality of primary probes each hybridized to the same target analyte thereby forming a complex. In some instances, the shrinking is applied to a complex of probes hybridized to a target.

Methods comprising shrinking the probe or product thereof may also be useful in embodiments where the target analyte is a non-nucleic acid target analyte. For example, the target analyte may be protein that is a member of a protein-protein interaction network. Shrinking a non-nucleic acid target analyte can be advantageous to pull apart such interaction networks to separate the analytes from one another and better resolve the interaction network.

In some embodiments, the probe comprises a protein target-binding moiety and a reporter oligonucleotide. In some embodiments, the protein target-binding moiety directly or indirectly binds to a protein target analyte. In some embodiments, the target-binding moiety is an antibody or epitope binding fragment thereof that directly or indirectly binds the protein target analyte.

In some embodiments, provided herein is a method for processing a biological sample, comprising: (a) contacting the biological sample with a polymer, wherein: the polymer comprises one or more functional groups A; the biological sample comprises a probe or a product thereof that comprises one or more functional groups B; and the probe comprises a nucleic acid and is configured to directly or indirectly bind to a protein target analyte in the biological sample; (b) reacting the one or more functional groups A with the one or more functional groups B, thereby coupling the polymer to the probe or product thereof to form a polymer conjugate; (c) removing unreacted polymer from the biological sample; and, (d) shrinking the polymer conjugate, thereby shrinking the probe or product thereof.

### E. Exemplary Embodiments of Expanding the Polymer Conjugate

The method may comprise expanding the polymer conjugate in the biological sample.

Expansion microscopy is an example of expanding polymer conjugates. Expansion microscopy expands all biomolecules inside a biological sample by adding a swellable (e.g., size controllable) polymer network, crosslinking the biomolecules to the polymer network, digesting the biological sample to break bonds between interacting biomolecules, and subsequently expanding the polymer network. The expansion of the polymer network expands the biological sample, thereby separating all biomolecules that have been crosslinked to the polymer network from one another. Expansion microcopy may be useful for separation of biomolecules for visualization using microscopy. Additional discussion of expansion microscopy can be found in, for example, U.S. Pat. Nos. 10,30,9879, 10,364,457, and 10,317,321, the contents of each of which are herein incorporated by reference in their entireties.

In some embodiments, the method comprises expanding a probe or product thereof in the biological sample, wherein the probe comprises a nucleic acid and is capable of binding with a target analyte in the biological sample. In some embodiments, the method comprises expanding the target analyte in the biological sample. Methods comprising expanding the probe(s) or product may be useful in embodiments where the target analyte is a nucleic acid target analyte. For example, the nucleic acid target analyte may be in an inaccessible region of DNA (e.g., a chromatin region). It can be advantageous to expand the probe or product thereof to open a chromatin region.

In some embodiments, the target nucleic analyte is a target nucleic acid is in a chromatin region. In some embodiments, the target analyte is a target nucleic acid in an open chromatin region, and expansion of the probe or product thereof opens the open chromatin region. In some embodiments, the target analyte is a target nucleic acid in a closed chromatin region, and expansion of the probe or product thereof opens the closed chromatin region.

In some embodiments, two or more target analytes may be analyzed, such as for detection of molecular interactions (e.g., protein-protein interactions). In some instances, the target analytes are close in proximity and expansion of probes bound to each target analyte of the two or more target analytes allows detection. In some embodiments, probes or products generated using the probes can be expanded from other probes or products.

In some aspects, provided herein is a method for processing a biological sample, comprising: (a) contacting the biological sample with a polymer, wherein: the polymer comprises one or more functional groups A; the biological sample comprises a probe or a product thereof that comprises one or more functional groups B; and the probe comprises a nucleic acid and is configured to directly or indirectly bind to a nucleic acid target analyte in the biological sample; (b) reacting the one or more functional groups A with the one or more functional groups B, thereby coupling the polymer to the probe or product thereof to form a polymer conjugate; (c) removing unreacted polymer from the biological sample; and, (d) expanding the polymer conjugate, thereby expanding the probe or product thereof. In some embodiments, the nucleic acid target analyte is in a chromatin region.

### III. Detection and Analysis

In some embodiments, the method further comprises detecting a signal associated with the probe or product thereof in the biological sample. In some embodiments, the method further comprises detecting the target analyte. Detecting may comprises sequence the target analyte, or a portion thereof (e.g., a barcode sequence), imaging the target analyte, or a combination thereof. In some embodiments, the method further comprises sequencing the target analyte. In some embodiments, the method further comprises detecting a signal associated with the target analyte. In some embodiments, less amplification is required for signal detection in a method that uses the methods provided herein for shrinking and/or expanding the polymer conjugate compared to a method wherein the biomolecule is not coupled to a polymer to form a polymer conjugate.

In some embodiments, a sequence of the probe or product thereof is analyzed in situ in the biological sample. In some embodiments, the sequence of the probe or product thereof is analyzed by sequential hybridization, sequencing by hybridization, sequencing by ligation, sequencing by synthesis, sequencing by binding, or a combination thereof. In some embodiments, the sequence that is analyzed comprises a barcode sequence. In some embodiments, the barcode sequence corresponds to the target analyte or a portion thereof or a labelling agent for the target analyte or a portion thereof in the biological sample. In some embodiments, the signal is detected by imaging the biological sample using fluorescent microscopy. In some embodiments, the signal is detected after the shrinking or expansion of the probe or product thereof. In some embodiments, the signal is detected during the shrinking or expansion of the probe or product thereof. In some embodiments, the signal is detected during and after the shrinking or expansion of the probe or product thereof.

In some embodiments, the target analyte is detected in situ in the biological sample. In some embodiments, the method comprises imaging the biological sample to detect the target analyte. In some embodiments, the imaging comprises fluorescent microscopy. In some embodiments, shrinking or expanding the polymer conjugate occurs during the imaging. In some embodiments, shrinking or expanding the polymer conjugate occurs before the imaging. In some embodiments, shrinking or expanding the polymer conjugate occurs before the imaging and continues during the imaging.

In some aspects, the provided methods involve analyzing, e.g., detecting or determining, one or more sequences present in the target analyte or associated with the target analyte. In some embodiments, the provided methods involve analyzing, e.g., detecting or determining, one or more sequences present in a target analyte described herein. In some embodiments, the detecting comprises hybridizing one or more detectably labeled probes to the probe or product thereof or the target analyte, or via hybridization to adaptor probes that hybridize to the probe or product thereof or the target analyte). In some embodiments, the analysis comprises determining the sequence of all or a portion of the probe or product thereof or the target analyte (e.g., a barcode sequence or a complement thereof), wherein the sequence is indicative of a sequence of the target analyte (e.g., a nucleic acid target analyte, such as a nucleic acid concatemer, e.g., an RCP).

In some cases, analysis is performed on one or more images captured, and may comprise processing the image(s) and/or quantifying signals observed. In some embodiments, images of signals from different fluorescent channels and/or detectable probe hybridization cycles can be compared and analyzed. In some embodiments, images of signals (or absence thereof) at a particular location in a sample from different fluorescent channels and/or sequential detectable probe hybridization cycles can be aligned to analyze an analyte at the location. For instance, a particular location in a sample can be tracked and signal spots from sequential probe hybridization (and optionally ligation) cycles can be analyzed to detect a target polynucleotide sequence (e.g., a barcode sequence or subsequence thereof) in a nucleic acid at the location. The analysis may comprise processing information of one or more cell types, one or more types of analytes, a number or level of analyte, and/or a number or level of cells detected in a particular region of the sample. In some embodiments, the analysis comprises detecting a sequence e.g., a barcode sequence present in an amplification product at a location in the sample. In some embodiments, the analysis includes quantification of puncta (e.g., if amplification products are detected). In some cases, the analysis includes determining whether particular cells and/or signals are present that correlate with one or more analytes from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization in a FISH-type assay, sequencing by hybridization).

In some embodiments, the methods comprise determining the sequence of all or a portion of a product of a probe hybridized to the target analyte (e.g., as described in Section II), such as one or more barcode sequences present in the product. In some embodiments, the sequence of the product, or barcode thereof, is indicative of a sequence of the target nucleic acid to which the product and probe is hybridized. In some embodiments, the analysis and/or sequence determination comprises sequencing all or a portion of the product and/or in situ hybridization to the product. In some embodiments, the sequencing step involves sequencing by hybridization, sequencing by ligation, sequencing by synthesis, sequencing by binding, and/or fluorescent in situ sequencing (FISSEQ), hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US2017/0009278, which is incorporated herein by reference, for exemplary probes and HCR reaction components. In some embodiments, the detection or determination comprises hybridizing to the first overhang a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is an amplification product (e.g., an RCP/nucleic acid concatemer).

In some embodiments, signal detection described herein can comprise signal amplification. Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method may be used.

In some embodiments, signal detection described herein can comprise assembly of branched nucleic acid complexes. In some embodiments, the assembly complex comprises an amplifier hybridized directly or indirectly (via one or more oligonucleotides) to a sequence of the primary probe or product thereof. In some embodiments, the assembly includes one or more amplifiers each including an amplifier repeating sequence. In some aspects, the one or more amplifiers is labelled. For exemplary branched signal amplification, see e.g., U.S. Pat. Pub. No. US 2020/0399689 A1 and Xia et al., Multiplexed Detection of RNA using MERFISH and branched DNA amplification. Scientific Reports (2019), each of which is fully incorporated by reference herein.

In some embodiments, signal detection described herein can comprise hybridization chain reaction (HCR). HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product. Exemplary methods and compositions for LO-HCR are described in US 2021/0198723, incorporated herein by reference in its entirety.

In some embodiments, signal detection described herein can comprise a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising probes or products thereof (e.g., as described in Section II). In various embodiments, the probes or products thereof may be contacted with a plurality of concatemer primers and a plurality of labelled probes. see e.g., U.S. Pat. Pub. No. US 2019/0106733, which is incorporated herein by reference, for exemplary molecules and PER reaction components.

In some embodiments, signal detection described herein can comprise a chain reaction that forms an amplification product, e.g., HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the probes (e.g., barcodes) or products thereof.

In some aspects, the provided methods comprise imaging the probe hybridized to the target analyte, for example, via binding of the secondary probe (e.g., a detectable probe) and detecting the detectable label. In some embodiments, the detectable probe comprises a detectable label that can be measured and quantitated. The terms “label” and “detectable label” comprise a directly or indirectly detectable moiety that is associated with (e.g., conjugated to) a molecule to be detected, e.g., a secondary probe that is a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more target analyte(s) described herein. In some embodiments, the methods involve incubating the detectable probe containing the detectable label with the sample, washing unbound detectable probe, and detecting the label, e.g., by imaging. In some embodiments, the target analyte(s) remain crosslinked to the target nucleic acid during the washing and detecting steps.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264.

As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores can include those described in Henegariu et al. (2000) Nature Biotechnol. 18:345, incorporated herein by reference.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as an Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, each of which is incorporated herein by reference in its entirety. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using microscopy or other imaging techniques described herein. In such aspects, the detecting comprises determining a signal, e.g., a fluorescent signal. In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detectable probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detectable probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932. Exemplary techniques for in situ sequencing or sequence detection comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121). References in this paragraph are incorporated herein by reference.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are incorporated herein by reference.

In some embodiments, sequence analysis of nucleic acids (e.g., nucleic acids such as RCA products comprising barcode sequences) can be performed by sequential hybridization (e.g., sequencing by hybridization and/or sequential in situ fluorescence hybridization). Sequential fluorescence hybridization can involve sequential hybridization of detectable probes comprising an oligonucleotide and a detectable label. In some embodiments, a method disclosed herein comprises sequential hybridization of the detectable probes disclosed herein, including detectably labelled probes (e.g., fluorophore conjugated oligonucleotides) and/or probes that are not detectably labelled per se but are capable of binding (e.g., via nucleic acid hybridization) and being detected by detectably labelled probes. Exemplary methods comprising sequential fluorescence hybridization of detectable probes are described in US 2019/0161796, US 2020/0224244, US 2022/0010358, US 2021/0340618, and US 2023/0039899A1, all of which are incorporated herein by reference in their entireties. In some embodiments, the methods provided herein can include analyzing the identifier sequences (e.g., analyte sequences or barcode sequences) by sequential hybridization and detection with a plurality of labelled probes (e.g., detection oligonucleotides).

In some embodiments, sequencing can be performed using sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597.

In some embodiments, the detectable probes are targeted by detectably labeled oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; US 2021/0017587 A1; US 2020/0080139 A1; US 2021/0017587 A1; and US 2017/0220733 A1, all of which are incorporated by reference in their entireties. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### IV. Samples, Analytes, and Target Sequences

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate as described elsewhere herein.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i). Disaggregation of Cells

In some embodiments, the biological sample corresponds to cells (e.g., derived from a cell culture, a tissue sample, or cells deposited on a surface). In a cell sample with a plurality of cells, individual cells can be naturally unaggregated. For example, the cells can be derived from a suspension of cells and/or disassociated or disaggregated cells from a tissue or tissue section.

Alternatively, the cells in the sample may be aggregated, and may be disaggregated into individual cells using, for example, enzymatic or mechanical techniques. Examples of enzymes used in enzymatic disaggregation include, but are not limited to, dispase, collagenase, trypsin, and combinations thereof. Mechanical disaggregation can be performed, for example, using a tissue homogenizer. The biological sample may comprise disaggregated cells (e.g., nonadherent or suspended cells) which are deposited on a surface and subjected to an in situ assay disclosed herein.

### (ii). Substrate Attachment

In some embodiments, the biological sample can be attached to a substrate. Examples of substrates suitable for this purpose are described in detail below. Attachment of the biological sample can be irreversible or reversible, depending upon the nature of the sample and subsequent steps in the analytical method.

In certain embodiments, the sample can be attached to the substrate reversibly by applying a suitable polymer coating to the substrate, and contacting the sample to the polymer coating. The sample can then be detached from the substrate using an organic solvent that at least partially dissolves the polymer coating. Hydrogels are examples of polymers that are suitable for this purpose.

More generally, in some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

(iii) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (iv) Isometric Expansion

In some embodiments, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016 and U.S. Pat. No. 10,059,990, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

### (v) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

### (vi) Tissue Permeabilization and Treatment

In some embodiments, a biological sample (e.g., dissociated cells on a substrate) can be permeabilized to facilitate transfer of species (such as labeling agents and/or detectable probes) into the sample. If a sample is not permeabilized sufficiently, the amount of analyte that is accessible to detecting reagents (e.g., labeling agents and/or detectable probes) may be too low to enable adequate analysis. Conversely, if the sample is too permeable, the relative spatial relationship of the analytes within the sample (e.g., dissociated cells on a substrate) can be lost. Hence, in some cases, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

B. Analytes

A biological sample may comprise one or a plurality of analytes of interest. Methods for performing multiplexed assays to analyze two or more different analytes in a single biological sample are provided.

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of an RCA (rolling circle amplification) template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g., including but not limited to protein-DNA complexes, which may or may not be covalently bound to one another, and which may be the same or different. Thus, in addition to cells or microorganisms, such a complex analyte may also be a protein complex or protein interaction. Such a complex or interaction may thus be a homo- or hetero-multimer. Aggregates of molecules, e.g., proteins may also be target analytes, for example aggregates of the same protein or different proteins. The analyte may also be a complex between proteins or peptides and nucleic acid molecules such as DNA or RNA, e.g., interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and epitope binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be derived from or detected in a live cell. Processing conditions can be adjusted to ensure that a biological sample remains live during analysis. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In some embodiments described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, a labeling agent may include an agent that interacts with an analyte (e.g., an endogenous analyte in a sample). In some embodiments, the labeling agents can comprise a binding moiety and a reporter oligonucleotide. The reporter oligonucleotide is indicative of the target analyte or portion thereof interacting with the binding moiety. For example, the reporter oligonucleotide may comprise a barcode sequence that permits identification of the binding moiety. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a reporter oligonucleotide comprising one or more barcode sequences, e.g., a barcode sequence that corresponds to the binding moiety and/or the target analyte. A binding moiety barcode or a target-specific barcode includes a barcode that is associated with or otherwise identifies the binding moiety. In some embodiments, by identifying a binding moiety by identifying its associated target-specific barcode, the target analyte to which the binding moiety binds can also be identified. In some embodiments, the labeling agent comprises one or more barcode sequences, e.g., a sample-specific barcode sequence that corresponds to the samples from which the biological cell is derived. The sample-specific or target-specific barcode can be a nucleic acid sequence of a given length and/or sequence that is associated with the binding moiety. A sample-specific or target-specific barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, a binding moiety may include any molecule or moiety capable of binding to an analyte (e.g., a biological analyte, e.g., a macromolecular constituent) which may be in and/or on a cell. A binding moiety may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The analyte binding moiety can be attached to a reporter oligonucleotide that is indicative of the sample from which a cell is derived. For instance, the same analyte binding moiety (e.g., an antibody or a lipid moiety configured to bind to the same cell surface feature) can be used to bind to cells from different samples, wherein the same analyte binding moiety is attached to different reporter oligonucleotides each identifying one of the different samples. For example, the reporter oligonucleotide may comprise a barcode sequence that permits identification of the sample from which a cell labeled with the reporter oligonucleotide is derived. For example, a first sample can be contacted with a binding moiety that is coupled thereto a first reporter oligonucleotide identifying the first sample, while a second sample can be contacted with the same binding moiety which is coupled to a different reporter oligonucleotide identifying the second sample.

Optionally, the reporter oligonucleotide can be indicative of a cell surface feature to which the binding group binds. For example, a binding moiety that is specific to one type of cell feature (e.g., a first cell surface feature) may have coupled thereto a first reporter oligonucleotide, while a binding moiety that is specific to a different cell feature (e.g., a second cell surface feature) may have a different reporter oligonucleotide coupled thereto. For a description of exemplary binding moieties, including antibody-based labeling agents, reporter oligonucleotides, and methods of use, see, e.g., U.S. Pat. No. 10,550,429; US2022/0228220 A1; U.S. Pat. Pub. 20190177800; and U.S. Pat. Pub. 20190367969, which are incorporated by reference herein in their entireties.

In some embodiments, a binding moiety includes one or more antibodies or epitope binding fragments thereof. The antibodies or epitope binding fragments including the binding moiety can specifically bind to a target analyte. In some embodiments, the analyte is a protein (e.g., a protein on a surface of the biological sample (e.g., a cell) or an intracellular protein). In some embodiments, a plurality of labeling agents comprising a plurality of binding moieties bind a plurality of analytes present in a biological sample. In some embodiments, the plurality of analytes includes a single species of analyte (e.g., a single species of polypeptide). In some embodiments in which the plurality of analytes includes a single species of analyte, the binding moieties of the plurality of labeling agents are the same. In some embodiments in which the plurality of analytes includes a single species of analyte, the binding moieties of the plurality of labeling agents are the different (e.g., members of the plurality of analyte labeling agents can have two or more species of binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the binding moiety (e.g., an antibody or lipophilic moiety) coupled to a first reporter oligonucleotide and a second plurality of the binding moiety coupled to a second reporter oligonucleotide.

In some aspects, these reporter oligonucleotides may comprise nucleic acid barcode sequences that permit identification of the sample origins of cells labeled with the reporter oligonucleotides. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the binding moiety may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or agents that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some instances, in addition to the sample-specific barcode sequence, a reporter oligonucleotide described herein may comprise one or more other sequences, such as an additional barcode sequence, an adapter sequence, a unique molecular identifier (UMI) sequence, and/or a primer or primer binding sequence.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the binding moiety or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by a labeling agent comprising a binding moiety that binds to the cell surface protein and a binding moiety barcode that identifies that binding moiety. Results of protein analysis in a sample (e.g., a tissue sample or a cell) can be associated with DNA and/or RNA analysis in the sample.

(iii) Products of Endogenous Analyte and/or Labeling Agent

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

(b) Ligation

In some embodiments, a product of an endogenous analyte and/or a labeling agent is a ligation product. In some embodiments, the ligation product is formed between two or more endogenous analytes. In some embodiments, the ligation product is formed between two or more labeling agent. In some embodiments, the ligation product is an intramolecular ligation of an endogenous analyte. In some embodiments, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation (e.g., click chemistry ligation). Exemplary click chemistry reactions are described in Section III-A-vi. In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(a) Hybridization

In some embodiments, a product of an endogenous analyte and/or a labeling agent is a hybridization product comprising the pairing of substantially complementary or complementary nucleic acid sequences within two different molecules, one of which is the endogenous analyte or the labeling agent (e.g., reporter oligonucleotide attached thereto). The other molecule can be another endogenous molecule or another labeling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Exemplary barcoded probes or probe sets may be based on a circularizable probe, a gapped circularizable probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

(c) Primer Extension and Amplification

In some embodiments, a product is a primer extension product of an analyte, a labeling agent, a probe or probe set bound to the analyte (e.g., as described in Section II), or a probe or probe set bound to the labeling agent (e.g., a probe bound to one or more reporter oligonucleotides from the same or different labeling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (i.e., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labeling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the RCA template may comprise the target analyte, or a part thereof, where the target analyte is a nucleic acid, or it may be provided or generated as a proxy, or a marker, for the analyte. In some embodiments, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (i.e. a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a nucleic acid probe disclosed herein may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe (e.g., a nucleic acid probe described herein). The exogenously added nucleic acid probe may be optionally ligated to a cellular nucleic acid molecule or another exogenous nucleic acid molecule. In other examples, a product comprising a target sequence for a nucleic acid probe disclosed herein may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In some embodiments, a detectable probe may comprise an overhang that does not hybridize to an RCA product but hybridizes to another probe (e.g., a fluorescently labeled probe).

C. Target Sequences

A target sequence for a probe disclosed herein may be comprised in any analyte disclose herein, including an endogenous analyte (e.g., a viral or cellular nucleic acid), a labeling agent, or a product of an endogenous analyte and/or a labeling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding embodiments, the methods provided herein can include analyzing the barcodes by sequential hybridization and detection with a plurality of labeled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4′ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and US20210164039A1, which are hereby incorporated by reference in their entireties.

### V. Compositions, Kits, and Systems

In some embodiments, disclosed herein is a composition that comprises a complex containing a target analyte (e.g., a target non-nucleic acid or a target nucleic acid, e.g., a target nucleic amplification product, such as a nucleic acid concatemer, e.g., an RCP) and a probe or product thereof comprising a functional group and a polymer comprising a functional group, e.g., any of the target analytes, probes or products thereof, and polymers described in Section III (e.g., polymer conjugates). In some embodiments, the complex further comprises a secondary probe (e.g., a detection probe), e.g., as described in Section III. In some embodiments, a polymer conjugated to the probe or product thereof and a second polymer conjugated to the probe or product thereof are cross-linked to each other thereby forming a polymer network. In some embodiments, the polymer exists as a polymer network prior to conjugation to the probe or product thereof. In some embodiments, the polymer conjugate may be shrunk, thereby shrinking the probe or product thereof. In other embodiments, the polymer conjugate may be expanded, thereby expanding the probe or product thereof.

Also provided herein are kits, for example comprising one or more polymers or polymer networks, e.g., any described in Section II, and instructions for performing the methods provided herein. In some embodiments, the kits further comprises a probe or product thereof, e.g., any described in Section II. In some embodiments, the kits further comprise one or more reagents required for one or more steps comprising hybridization, ligation, extension, detection, sequencing, and/or sample preparation as described herein. In some embodiments, any or all of the probes or products thereof are DNA molecules.

In some embodiments, a kit disclosed herein can comprise a buffer that promotes shrinking of a polymer conjugate or network disclosed herein. In some embodiments, a kit disclosed herein can comprise a buffer that comprises at least or about 50% H₂O, at least or about 55% H₂O, at least or about 60% H₂O, at least or about 65% H₂O, at least or about 70% H₂O, at least or about 75% H₂O, at least or about 80% H₂O, at least or about 85% H₂O, at least or about 90% H₂O, or at least or about 95% H₂O.

In some embodiments, a kit disclosed herein can comprise a buffer that allows or promotes polymer conjugate or network expanding or loosening. In some embodiments, a kit disclosed herein can comprise a buffer composition comprising DMSO, H₂O, and a detergent or surfactant. In some embodiments, the buffer composition for polymer conjugate or network expanding or loosening comprises DMSO, H₂O, and a salt. In some embodiments, the buffer composition for polymer conjugate or network expanding or loosening comprises DMSO, H₂O, a detergent or surfactant, and a salt. In some embodiments, the buffer composition for polymer conjugate or network expanding or loosening comprises at least or about 50% DMSO, at least or about 55% DMSO, at least or about 60% DMSO, at least or about 65% DMSO, at least or about 70% DMSO, at least or about 75% DMSO, at least or about 80% DMSO, at least or about 85% DMSO, at least or about 90% DMSO, or at least or about 95% DMSO.

In some embodiments, a kit disclosed herein can comprise a buffer used during imaging, for instance, to detect fluorescent signals associated with nucleic acid probes that hybridize to RCPs in a sample. In some embodiments, the buffer maintains the shrunk polymer conjugate or polymer network which keeps the RCPs shrunk. In some embodiments, a kit disclosed herein can comprise a buffer comprising a phenolic acid, such as a dihydroxybenzoic acid. In some embodiments, the buffer that maintains the shrunk polymer conjugate or network comprises protocatechuic acid (PCA). In some embodiments, the buffer that maintains the shrunk polymer conjugate or network comprises a non-zwitterionic buffer. In some embodiments, the buffer that maintains the shrunk polymer conjugate or network comprises 1,3-bis(tris(hydroxymethyl)methylamino)propane, also known as BTP or BIS-TRIS propane. In some embodiments, the buffer comprises a dihydroxybenzoic acid and a non-zwitterionic buffer, such as PCA and BIS-TRIS propane, respectively, and the content and ratio of the dihydroxybenzoic acid (e.g., PCA) and non-zwitterionic buffer (e.g., BIS-TRIS propane) can be tuned depending on the need to keep the polymer conjugate or polymer network in a particular shrunk or expanded state, for instance, during imaging.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers. In some aspects, the kit can also comprise any of the reagents described herein, e.g., wash buffer, stripping buffer, etc. In some embodiments, the kits contain reagents for detection and/or sequencing, such as detectable probes or detectable labels. In some embodiments, the kits optionally contain other components, for example nucleic acid primers, enzymes and reagents, buffers, nucleotides, and reagents for additional assays.

### VI. Applications

In some aspects, the provided embodiments can be applied in an in situ method of processing a biological sample. For example, in some aspects, the provided embodiments can be applied in an in situ method of analyzing target nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to shrink nucleic acid concatemers via polymers that may be conjugated with a probe or product thereof in the biological sample, to increase the resolution and stability of the nucleic acid concatemers in situ. In some aspects, the provided embodiments can be used to shrink target analytes via polymers that may be conjugated with a target analyte in the biological sample, to increase the resolution and stability of the analytes in situ.

The methods herein have particular applicability in the detection of identifier sequences (e.g., analyte sequences or barcode sequences) in situ in a biological sample, including those using sequential cycles of detectable probe hybridization to decode the identifier sequences. In some embodiments, an identifier sequence herein comprises an analyte sequence, an analyte-derived sequence, or a complement thereof. In some embodiments, the analyte comprises a nucleic acid sequence, and an identifier sequence comprises the nucleic acid sequence in the analyte or a complement of the nucleic acid sequence. In some embodiments, an identifier sequence herein comprises a barcode sequence that is associated with, corresponds to, and/or identifies an analyte.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VII. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

(i) Barcode

A “barcode” is a label, or identifier, that conveys or is capable of conveying information (e.g., information about an analyte in a sample, a target, a bead, and/or a sample). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

(ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G).

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

(iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

(v) Subject

A “subject” is an animal, such as a mammal (e.g., human or a non-human simian), or avian (e.g., bird), or other organism, such as a plant. Examples of subjects include, but are not limited to, a mammal such as a rodent, mouse, rat, rabbit, guinea pig, ungulate, horse, sheep, pig, goat, cow, cat, dog, primate (e.g. human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, or honey bee; an arachnid such as a spider; a fish such as zebrafish; a reptile; an amphibian such as a frog or Xenopus laevis; a Dictyostelium discoideum; a fungi such as Pneumocystis carinii, Takifugu rubripes, yeast, Saccharomyces cerevisiae or Schizosaccharomyces pombe; or a Plasmodium falciparum.

(vi) Splint Oligonucleotide

A “splint oligonucleotide” is an oligonucleotide that, when hybridized to other polynucleotides, acts as a “splint” to position the polynucleotides next to one another so that they can be ligated together. In some embodiments, the splint oligonucleotide is DNA or RNA. The splint oligonucleotide can include a nucleotide sequence that is partially complimentary to nucleotide sequences from two or more different oligonucleotides. In some embodiments, the splint oligonucleotide assists in ligating a “donor” oligonucleotide and an “acceptor” oligonucleotide. In general, an RNA ligase, a DNA ligase, or another other variety of ligase is used to ligate two nucleotide sequences together.

In some embodiments, the splint oligonucleotide is between 10 and 50 oligonucleotides in length, e.g., between 10 and 45, 10 and 40, 10 and 35, 10 and 30, 10 and 25, or 10 and 20 oligonucleotides in length. In some embodiments, the splint oligonucleotide is between 15 and 50, 15 and 45, 15 and 40, 15 and 35, 15 and 30, 15 and 30, or 15 and 25 nucleotides in length.

(vii) Adaptor, Adapter, and Tag

An “adaptor,” an “adapter,” and a “tag” are terms that are used interchangeably in this disclosure, and refer to species that can be coupled to a polynucleotide sequence (in a process referred to as “tagging”) using any one of many different techniques including (but not limited to) ligation, hybridization, and tagmentation. Adaptors can also be nucleic acid sequences that add a function, e.g., spacer sequences, primer sequences/sites, barcode sequences, unique molecular identifier sequences.

(viii) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

(ix) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(x) Primer Extension

Two nucleic acid sequences can become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(xi) Proximity Ligation

A “proximity ligation” is a method of ligating two (or more) nucleic acid sequences that are in proximity with each other through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference).

A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(xii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence can be used as a template for single-strand synthesis of a corresponding cDNA molecule.

(xiii) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments, the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oN™ DNA ligase, available from New England Biolabs, Ipswich, MA), and Ampligase™ (available from Epicentre Biotechnologies, Madison, WI). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA can be carried out by real-time PCR (also known as quantitative PCR or qPCR), using techniques such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

(xiv) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form epitope binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the epitope binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)₂ fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific epitope binding ability to the polypeptide.

(xv) Affinity Group

An “affinity group” is a molecule or molecular moiety which has a high affinity or preference for associating or binding with another specific or particular molecule or moiety. The association or binding with another specific or particular molecule or moiety can be via a non-covalent interaction, such as hydrogen bonding, ionic forces, and van der Waals interactions. An affinity group can, for example, be biotin, which has a high affinity or preference to associate or bind to the protein avidin or streptavidin. An affinity group, for example, can also refer to avidin or streptavidin which has an affinity to biotin. Other examples of an affinity group and specific or particular molecule or moiety to which it binds or associates with include, but are not limited to, antibodies or antibody fragments and their respective antigens, such as digoxigenin and anti-digoxigenin antibodies, lectin, and carbohydrates (e.g., a sugar, a monosaccharide, a disaccharide, or a polysaccharide), and receptors and receptor ligands.

Any pair of affinity group and its specific or particular molecule or moiety to which it binds or associates with can have their roles reversed, for example, such that between a first molecule and a second molecule, in a first instance the first molecule is characterized as an affinity group for the second molecule, and in a second instance the second molecule is characterized as an affinity group for the first molecule.

(xvi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a detectable probe. For example, detectable labels can be incorporated during nucleic acid polymerization or amplification (e.g., Cy5®-labeled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, C1-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLES

The following examples are included for illustrative purposes only and is not intended to limit the scope of the present disclosure.

### Example 1: In Situ Targeted Shrinking of a Biomolecule Using a Pre-Crosslinked Polymer Network

This example demonstrates a method for in situ shrinking of a biomolecule (e.g., a nucleic acid concatemer or a protein) using a pre-crosslinked size-controllable polymer in a biological sample. In particular, this example demonstrates shrinking of a biomolecule via coupling a probe or product thereof to a cross-linked polymer network in situ in a biological sample, followed by targeted shrinking of the polymer conjugate. The probe comprises a nucleic acid and is capable of binding to the biomolecule in the biological sample.

FIG. 1 shows an exemplary workflow for shrinking a biomolecule (e.g., a probe or product thereof bound to the biomolecule). In step (i) of FIG. 1, a biological sample (e.g., a processed or cleared biological sample, a tissue sample, a sample embedded in a hydrogel, etc.) comprising a probe or product thereof 102 is contacted with a polymer 104. The probe or product thereof 102 comprises one or more functional groups B. In some examples, the one or more functional groups B are in one or more nucleotides on a nucleic acid target analyte, such as a nucleic acid product, e.g., an amplification product, e.g., a rolling circle amplification (RCA) product (RCP). In other examples, the one or more functional groups B may be in a probe bound to the target analyte. The probe comprises a nucleic acid, and is configured to directly or indirectly bind to the target analyte in the biological sample. The target analyte may be a non-nucleic acid target analyte (e.g., a protein) and the probe may be an antibody or epitope binding fragment thereof. Alternatively, the target analyte may be a nucleic acid target analyte, such as a nucleic acid product, e.g., an amplification product, e.g., an RCP.

The polymer 104 comprises one or more functional groups A, and is pre-crosslinked in the form a polymer network 106. The cross-linked polymer network may be formed by ion-ion interactions, hydrogen bonding, photo-crosslinking of the polymers, chemically crosslinking the polymers using a crosslinking reagent, and/or by a click reaction between the polymers. The one or more functional groups A of the polymer 104 are reacted with the one or more functional groups B of the probe or product thereof 102, thereby coupling the polymer to the probe or product thereof to form a polymer conjugate 108. In some examples, the reaction between functional group A and functional group B forms a covalent bond. For example, the reaction between functional group A and functional group B may be a click reaction, in particular a click reaction that is biorthogonal.

Unreacted polymer can then be removed from the biological sample. Removing the unreacted polymer can be optionally performed by washing the sample.

In some examples, the polymers of the polymer conjugate are size-controllable polymers. The polymer conjugate 108 is subsequently shrunk as shown in step (ii) of FIG. 1, resulting in a shrunken probe or product thereof 110. The shrinking may be initiated chemically, by changing the temperature of the biological sample, and/or by exposing the biological sample to light.

Overall, coupling a probe or product thereof with a pre-crosslinked polymer network allows for shrinking of the biomolecule. This process provides an improved method for analyzing a biological sample comprising a biomolecule.

### Example 2: In Situ Targeted Shrinking of a Biomolecule Using a Polymer

This example demonstrates a method for in situ shrinking of a biomolecule (e.g., a nucleic acid concatemer or a protein) using a size-controllable polymer in a biological sample. In particular, this example demonstrates shrinking of a biomolecule via coupling a probe or product thereof to a polymer network in situ in a biological sample, followed by targeted shrinking of the polymer conjugate. The probe comprises a nucleic acid and is capable of binding to the biomolecule in the biological sample.

FIG. 2 shows an exemplary workflow for shrinking of a biomolecule (e.g., a probe or product thereof bound to the biomolecule). In step (i) of FIG. 2, a biological sample (e.g., a processed or cleared biological sample, a tissue sample, a sample embedded in a hydrogel, etc.) comprising a probe or product thereof 202 is contacted with a polymer 204. The probe or product thereof 202 comprises one or more functional groups B. In some examples, the one or more functional groups B are in one or more nucleotides on a nucleic acid target analyte, such as a nucleic acid product, e.g., an amplification product, e.g., an RCP. In other examples, the one or more functional groups B may be in a probe bound to the target analyte. The probe comprises a nucleic acid, and is configured to directly or indirectly bind to the target analyte in the biological sample. The target analyte may be a non-nucleic acid target analyte (e.g., a protein) and the probe may be an antibody or epitope binding fragment thereof. Alternatively, the target analyte may be a nucleic acid target analyte, such as a nucleic acid product, e.g., an amplification product, e.g., an RCP.

The polymer 204 comprises one or more functional groups A. The one or more functional groups A of the polymer 204 are reacted with the one or more functional groups B of the nucleic acid 202 in step (ii) of FIG. 2, thereby coupling the polymer to the probe or product thereof to form a polymer conjugate 206. In some examples, the reaction between functional group A and functional group B forms a covalent bond. For example, the reaction between functional group A and functional group B may be a click reaction, in particular a click reaction that is biorthogonal. Unreacted polymer may be removed from the biological sample. Removing the unreacted polymer can be optionally performed by washing the sample.

As shown in step (iii) of FIG. 2 polymers of the polymer conjugate are cross-linked to form a polymer network conjugated nucleic acid 208. The cross-linked polymer network may be formed by ion-ion interactions, hydrogen bonding, photo-crosslinking of the polymers, chemically crosslinking the polymers using a crosslinking reagent, and/or by a click reaction between the polymers. In some examples, unreacted polymer is removed from the biological sample after the polymer-polymer crosslinking reaction.

In some examples, the polymers of the polymer conjugate are size-controllable polymers. The polymer conjugate 206 is subsequently shrunk as shown in step (iv) of FIG. 2, resulting in a shrunken probe or product thereof 210. The shrinking may be initiated by a stimulus. The shrinking may be initiated chemically, by changing the temperature of the biological sample, and/or by exposing the biological sample to light.

### Example 3: In Situ Targeted Expansion of a Biomolecule Using a Pre-Crosslinked Polymer Network

This example demonstrates a method for in situ expansion of a biomolecule (e.g., a nucleic acid target analyte in a chromatin region) using a pre-crosslinked size-controllable polymer in a biological sample. In particular, this example demonstrates expansion of a biomolecule via coupling a probe or product thereof to a cross-linked polymer network in situ in a biological sample, followed by targeted expansion of the polymer conjugate. The probe comprises a nucleic acid and is capable of binding to the biomolecule in the biological sample.

FIG. 3 shows an exemplary workflow for expanding a biomolecule (e.g., a probe or product thereof bound to the biomolecule). In step (i) of FIG. 3, a biological sample (e.g., a processed or cleared biological sample, a tissue sample, a sample embedded in a hydrogel, etc.) comprising a probe or product thereof 302 is contacted with a polymer 304. The probe or product thereof 302 comprises one or more functional groups B. In some examples, the one or more functional groups B are in one or more nucleotides on a nucleic acid target analyte, such as a nucleic acid in an inaccessible region of DNA, e.g., an open or closed chromatin region. In other examples, the one or more functional groups B may be in a probe bound to the target analyte. The probe comprises a nucleic acid, and is configured to directly or indirectly bind to the target analyte in the biological sample. The target analyte may be a nucleic acid target analyte, such as a nucleic acid product, e.g., an amplification product, such as a nucleic acid in an inaccessible region of DNA, e.g., an open or closed chromatin region.

The polymer 304 comprises one or more functional groups A, and is pre-crosslinked in the form a polymer network 306. The cross-linked polymer network may be formed by ion-ion interactions, hydrogen bonding, photo-crosslinking of the polymers, chemically crosslinking the polymers using a crosslinking reagent, and/or by a click reaction between the polymers. The one or more functional groups A of the polymer 304 are reacted with the one or more functional groups B of the probe or product thereof 302, thereby coupling the polymer to the probe or product thereof to form a polymer conjugate 308. In some examples, the reaction between functional group A and functional group B forms a covalent bond. For example, the reaction between functional group A and functional group B may be a click reaction, in particular a click reaction that is biorthogonal.

Unreacted polymer is then be removed from the biological sample. Removing the unreacted polymer can be optionally performed by washing the sample.

In some examples, the polymers of the polymer conjugate are size-controllable polymers. The polymer conjugate 308 is subsequently expanded as shown in step (ii) of FIG. 3, resulting in an expanded probe or product thereof 310. The expansion may be initiated chemically, by changing the temperature of the biological sample, and/or by exposing the biological sample to light.

Overall, coupling a probe or product thereof with a pre-crosslinked polymer network allows for expanding of the biomolecule. This process provides an improved method for analyzing a biological sample comprising a biomolecule.

### Example 4: In Situ Targeted Expansion of a Biomolecule Using a Polymer

This example demonstrates a method for in situ expansion of a biomolecule (e.g., a nucleic acid target analyte in a chromatin region) using a size-controllable polymer in a biological sample. In particular, this example demonstrates expansion of a biomolecule via coupling a probe or product thereof to a polymer network in situ in a biological sample, followed by targeted expansion of the polymer conjugate. The probe comprises a nucleic acid and is capable of binding to the biomolecule in the biological sample.

FIG. 4 shows an exemplary workflow for expanding a biomolecule (e.g., a probe or product thereof bound to the biomolecule). In step (i) of FIG. 4, a biological sample (e.g., a processed or cleared biological sample, a tissue sample, a sample embedded in a hydrogel, etc.) comprising a probe or product thereof 402 is contacted with a polymer 404. The probe or product thereof 202 comprises one or more functional groups B. In some examples, the one or more functional groups B are in one or more nucleotides on a nucleic acid target analyte, such as a nucleic acid in an inaccessible region of DNA, e.g., an open or closed chromatin region. In other examples, the one or more functional groups B may be in a probe bound to the target analyte. The probe comprises a nucleic acid, and is configured to directly or indirectly bind to the target analyte in the biological sample. The target analyte may be a nucleic acid target analyte, such as a nucleic acid product, e.g., an amplification product, such as a nucleic acid in an inaccessible region of DNA, e.g., an open or closed chromatin region.

The polymer 404 comprises one or more functional groups A. The one or more functional groups A of the polymer 404 are reacted with the one or more functional groups B of the probe or product thereof 402 in step (ii) of FIG. 4, thereby coupling the polymer to the probe or product thereof to form a polymer conjugate 406. In some examples, the reaction between functional group A and functional group B forms a covalent bond. For example, the reaction between functional group A and functional group B may be a click reaction, in particular a click reaction that is biorthogonal. Unreacted polymer may be removed from the biological sample. Removing the unreacted polymer can be optionally performed by washing the sample.

As shown in step (iii) of FIG. 4 polymers of the polymer conjugate are cross-linked to form of polymer network 408. The cross-linked polymer network may be formed by ion-ion interactions, hydrogen bonding, photo-crosslinking of the polymers, chemically crosslinking the polymers using a crosslinking reagent, and/or by a click reaction between the polymers. In some examples, unreacted polymer is removed from the biological sample after the polymer-polymer crosslinking reaction.

In some examples, the polymers of the polymer conjugate are size-controllable polymers. The polymer conjugate 406 is subsequently expanded as shown in step (iv) of FIG. 4, resulting in an expanded probe or product thereof 410. The expansion may be initiated chemically, by changing the temperature of the biological sample, and/or by exposing the biological sample to light.

In any of the examples provided, expansion and shrinking of the polymer network may be performed in combination in sequential steps in a reversible manner. In one example, a workflow for analyzing a biological sample comprises: contacting the sample with a plurality of primary probes for hybridizing to target sequences in the target nucleic acids of the biological sample; generating probe products using the primary probes wherein a probe product or complex thereof comprises one or more functional groups B, contacting the biological sample with a polymer, wherein the polymer comprises one or more functional groups A; reacting the one or more functional groups A with the one or more functional groups B, thereby coupling the polymer to the probe or product thereof to form a polymer conjugate; removing unreacted polymer from the biological sample. Once the polymer conjugate is formed in the polymer matrix, the biological sample can be contacted with one or more detectably labeled probes that hybridize directly or indirectly (e.g., via intermediate probes) to the primary probe or generated products thereof or the target analyte and a signal associated with the probe or product thereof or the target analyte can be detected (e.g., using fluorescent microscopy). The workflow includes cycles of contacting the biological sample with pools of the one or more detectably labeled probes (and optionally intermediate probes), detecting signals associated with the probe or product thereof or the target analyte, and removing the one or more detectably labeled probes before another cycle is repeated with another pool of detectably labeled probes. In some instances, shrinking or expanding the polymer network can be performed with the cycles of hybridizing the detectably labeled probe (and optionally intermediate probe) and detecting signals. For example, during probe hybridization (e.g., detectably labeled probes and/or intermediate probes), the polymer network can be expanded as described in Example 3 and 4, and during detection (e.g., imaging), shrinking can be performed as described in Example 1 and 2. By switching the buffer, shrinking of the polymer conjugate in the network during the imaging step may allow improved signal detection (e.g., brighter and more compact signals) and expanding of the polymer conjugate in the network during probe hybridization may allow improved diffusion and accessibility of the probes to the complementary sequences.

### Example 5: Modeling Relationship Between RCP Detection Intensity Vs RCP Sizes in Different Hydrogel Embedding Conditions

This example presents a model for investigating the relationship between the size of a biomolecule such as an amplification product (e.g., rolling circle amplification (RCA) product (RCP)) in a tissue sample and the intensity of signals that can be observed to detect the amplification product. To detect the RCP, detectably labeled probes were provided to bind directly or indirectly (e.g., via an intermediate probe) to the RCP generated using a circularized probe (e.g., a ligated probe) bound to a target analyte. The intensity of signals that can be detected from the detectably labeled probes bound to the RCP may depend on factors including, for example, the size of the amplification product, the conformation of the amplification product, the accessibility of probes (e.g., detectably labeled probes) to bind to the amplification product and factors related to the label on the detectably labeled probes, such as the quantum yield of the fluorescent dye (e.g., level of quenching of the dye). The intensity (I) is proportional to the number of fluorescent dyes, which equals the number of detected labeled probes bound to the intermediate probes bound to the RCP. The intensity can be determined based on:

I˜# of fluorescent dyes=# of detectably labeled probes bound to intermediate probes bound to the RCP˜# of nucleotides (N) in the RCP

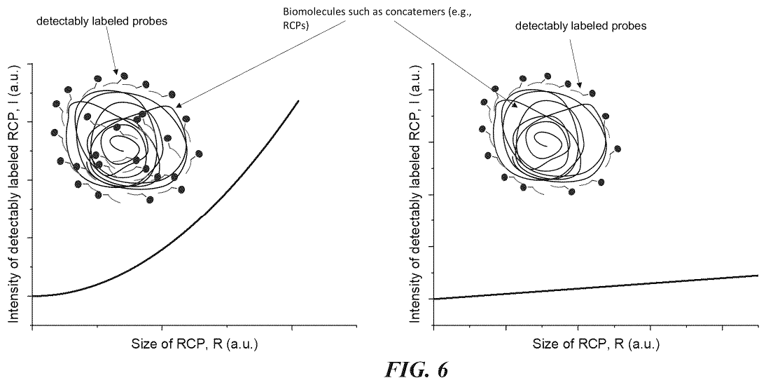

Assuming each RCP is a concatemer with secondary structure (e.g., a coil), then radius of the RCP, R, the radius of gyration of the coil, is proportional to square root of N. The relationship is as follows R∝N\hat{ }(½)*b, where b is the average size of each nucleotide in the RCP. In an ideal case, where every nucleotide of the RCP (i.e., every nucleotide available for binding) is accessible to the detectably labeled probes, then I should be I˜R\hat{ }2, as shown in the left side of FIG. 6. However, if only the surface of the RCP is accessible to detectably labeled probes, then the surface area of the RCP∝N and thus # of detectably labeled probes∝# of nucleotides, e.g., the observed I˜R (right side of FIG. 6). The illustrated RCP coil and detectably labeled probes on the left depict that probes have full access to bind the RCP, where I˜R\hat{ }2. The illustrated RCP coil and detectably labeled probes on the right depict that probes only have access to bind the surface of the RCP, where I˜R. Thus, fitting I as a function of N with a power law, I˜N\hat{ }α, can provide insight on the accessibility of the probes. A higher a indicates higher accessibility and a lower a suggests that the RCP is not entirely accessible and that one or more of the factors described above can play a role in the reduced intensity of signals observed.

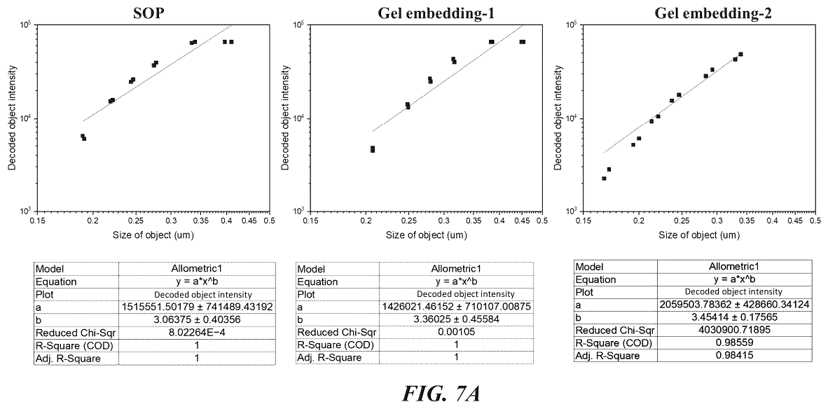

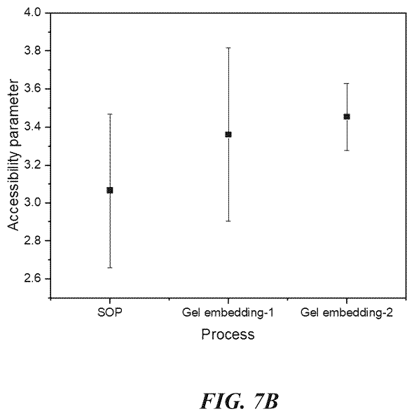

As shown in the plots FIG. 7A-7B, power of the fitting indicates the combinatorial effect of the factors. In these graphs, the higher the power value indicates that there is higher “accessibility” to the amplification product to bind detectably labeled probes for generating a signal. In a sample where the RCPs were not tethered and the tissue was not embedded in a hydrogel matrix (SOP), the a was 3.06. For comparison, in both gel embedded cases (gel embedding-1 and gel embedding-2) the a parameters were 10% and 15% higher than SOP, respectively. In one condition (gel embedding-1), the sample was embedded in the hydrogel matrix after ligation of circularizable probes but before performing RCA. In another condition (gel-embedding-2), the sample was embedded in the hydrogel matrix after ligation of the circularizable probes but before performing RCA and the sample was treated with proteinase K for 3 minutes.

From comparing the various sample treatment and embedding conditions, the relationship between the intensity of the detected objects and size of the objects shown in FIG. 7A-7B indicate that the RCPs in these conditions may not be entirely accessible to detectably labeled probes. To increase accessibility to binding sites in the RCPs, the methods described herein can be employed to achieve targeted expansion of a biomolecule (e.g., RCPs in a sample) by coupling a probe or product thereof to a polymer network, restoring accessibility to binding detectably labeled probes.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for processing a biological sample, comprising:
(a) contacting the biological sample with a polymer, wherein:
the polymer comprises one or more functional groups A;
the biological sample comprises a probe or a product thereof that comprises one or more functional groups B; and
the probe comprises a nucleic acid and is configured to directly or indirectly bind to a target analyte in the biological sample;
(b) reacting the one or more functional groups A with the one or more functional groups B, thereby coupling the polymer to the probe or product thereof to form a polymer conjugate;
(c) removing unreacted polymer from the biological sample; and
(d) shrinking or expanding the polymer conjugate, thereby respectively shrinking or expanding the probe or product thereof.

2. (canceled)

3. The method of claim 1, further comprising initiating polymer-polymer cross-linking to form a polymer network between the polymers of the polymer conjugate.

4. (canceled)

5. The method of claim 1, wherein the reaction between functional group A and functional group B is a click reaction.

6-10. (canceled)

11. The method of claim 1, wherein the target analyte is a nucleic acid target analyte, and wherein a primary probe is hybridized to a target sequence in the nucleic acid target analyte.

12-14. (canceled)

15. The method of claim 11, wherein the product of the primary probe is a rolling circle amplification (RCA) product (RCP) and nucleotides comprising one or more functional groups B are incorporated into the RCP during RCA, and wherein the RCP is shrunk.

16-21. (canceled)

22. The method of claim 1, wherein the probe or product thereof comprises a detectable label, or a region that directly or indirectly binds to a detectably labeled probe.

23-27. (canceled)

28. The method of claim 22, wherein the region in the probe or product thereof comprises a barcode sequence, and wherein the barcode sequence corresponds to an analyte or a portion thereof in the biological sample.

29. The method of claim 1, further comprising detecting a signal associated with the probe or product thereof or the target analyte by imaging the biological sample using fluorescent microscopy, after the shrinking or expansion of the probe or product thereof.

30-31. (canceled)

32. The method of claim 29, wherein the signal is detected after the shrinking of the probe or product thereof and the polymer conjugate is expanded after the signal is detected, thereby expanding the probe or product thereof.

33. The method of claim 32, wherein the polymer conjugate is expanded in a buffer composition comprising at least or about 50% DMSO, a detergent or surfactant, and a salt.

34-44. (canceled)

45. The method of claim 1, wherein shrinking or expanding the polymer conjugate is initiated by contacting the biological sample with a solution, by changing a temperature of the biological sample, and/or by exposing the biological sample to light.

46-51. (canceled)

52. The method of claim 1, wherein the biological sample is a tissue section.

53. The method of claim 1, wherein the biological sample is a processed or cleared biological sample.

54-78. (canceled)

79. The method of claim 1, wherein contacting the biological sample with the polymer occurs after generation of the nucleic acid product of the probe in the biological sample.

80. The method of claim 1, wherein the nucleic product of the probe is generated in situ in the biological sample.

81-84. (canceled)

85. The method of claim 1, wherein the nucleic product of the probe is a rolling circle amplification (RCA) product (RCP) of a circular or circularizable probe or probe set that hybridizes to a DNA or RNA molecule in the biological sample.

86-93. (canceled)

94. The method of claim 85, further comprising degrading the polymer conjugate after shrinking the nucleic acid product and detecting a signal associated with the product of the probe in the biological sample.

95. The method of claim 94, wherein the degrading comprises contacting the biological sample with a stripping buffer, changing the temperature of the biological sample, and/or exposing the biological sample to light.

96-108. (canceled)

109. A method, comprising:
(a) generating a rolling circle amplification product (RCP) at a location in a biological sample;
(b) contacting the biological sample with a polymer, wherein:
the polymer comprises one or more functional groups A, and
the RCP comprises one or more functional groups B;
(c) reacting the one or more functional groups A with the one or more functional groups B, thereby coupling the polymer to the RCP to form a polymer conjugate;
(d) removing unreacted polymer from the biological sample;
(e) shrinking the polymer conjugate, thereby shrinking the RCP; and
(f) detecting a signal associated with the RCP by imaging the biological sample using fluorescent microscopy, thereby detecting the RCP at the location in the biological sample.

110. The method of claim 109, further comprising:
(g) expanding the polymer conjugate after the signal is detected in (f), thereby expanding the RCP.
